(19) United States
(12) Patent Application Publication
(10) Pub. No.: US 2026/0245664 A1
(43) Pub. Date: Aug. 20, 2026

(54) METHODS AND SYSTEMS FOR DETECTING HOMOLOGOUS RECOMBINATION DEFICIENCY IN CANCER THERAPIES

(71) Applicant: The Regents of the University of California, Oakland, CA (US)

(72) Inventors: Ludmil Boyanov Alexandrov, San Diego, CA (US); Ammal Abbasi, La Jolla, CA (US)

(21) Appl. No.: 18/874,569

(22) PCT Filed: Jun. 14, 2023
(86) PCT No.: PCT/US2023/068465

§ 371 Date: Dec. 12, 2024

Related U.S. Application Data
(60) Provisional application No. 63/366,392, filed on Jun. 14, 2022.

(51) Int. Cl. G16B 40/20; A61K 31/282; A61K 31/4184; A61K 31/454; A61K 31/502; A61K 31/5025; A61K 31/55; A61K 33/243; G16B 20/00; G16H 20/10

(52) U.S. Cl. CPC G16B 40/20; A61K 31/282; A61K 31/4184; A61K 31/454; A61K 31/502; A61K 31/5025; A61K 31/55; A61K 33/243; G16B 20/00; G16H 20/10

(57) ABSTRACT

Provided are methods and systems related to detection of homologous recombination deficiency. Methods of generating a homologous recombination feature set, training a predictive model configured to predict the presence of homologous recombination deficiency in a subject, and administering a cancer therapeutic to a subject are specified. A computer system configured to output a homologous recombination classification of a subject is also specified. The methods and system are applicable to both whole-exome and whole-genome sequencing data.

### CROSS-REFERENCE TO RELATED APPLICATIONS

This application claims the benefit of U.S. Provisional Patent Application No. 63/366,392 filed on Jun. 14, 2022, the contents of which are incorporated by reference in their entirety.

### TECHNICAL FIELD

The present technology relates to methods of generating a homologous recombination feature set, methods of training a predictive model to predict the presence of homologous recombination deficiency, and systems configured to output a homologous recombination classification. The present technology also relates to methods of administering cancer therapeutics to a subject.

### BACKGROUND

The repair of DNA double strand breaks by homologous recombination (HR) is an essential cellular mechanism for maintaining genomic stability and preventing tumorigenesis. Prior studies have elucidated key genes in the HR pathway, e.g., BRCA1, BRCA2, RAD51, and PALB2, that commonly exhibit germline or somatic mutations observed in breast, ovarian, and pancreatic cancers.

Defects in HR genes can disable the HR repair pathway, making cells vulnerable to double strand breaks, and thus providing a treatment opportunity. Specifically, cancer patients prone to defective HR repair may be sensitive to poly (ADPribose) polymerase (PARP) inhibitors and/or platinum therapies. PARP inhibitors induce double strand breaks by stalling the replication fork during DNA replication, thereby increasing the reliance on error-prone alternative repair pathways in HR deficient (HRD) cells, causing the cell to accumulate mutations and to consequently undergo apoptosis. Likewise, platinum therapies cause inter-strand breaks, leading to p53-initiated apoptosis in HRD cells.

Conventional stratification of HR deficient patients (HRD patients) involves screening for canonical genomic markers including pathogenic germline variants and somatic copy number alterations in HR genes. Two commercial HRD companion diagnostic (CDx) tests, Myriad myChoice® CDx and FoundationOne® CDx, have been approved by the U.S. Food and Drug Administration (FDA) for patients with ovarian cancer. Myriad myChoice® CDx and FoundationOne® CDx both determine HRD by quantifying overall genomic instability in combination with BRCA1 and BRCA2 status.

In addition, at least three academic approaches, SigMA, HRDetect, and CHORD, have been developed to capture HR deficient cancers by applying machine learning approaches to study the patterns of somatic mutations found in cancer sequencing data. For example, SigMA was specifically developed to detect SBS3, a mutational signature of single base substitutions (SBS) previously attributed to HRD. Unfortunately, SigMA is only applicable to targeted panel and whole-exome sequencing data from highly mutated cancers (<15% of all breast, ovarian, and pancreatic cancers). HRDetect is a machine learning tool that detects HR deficient cancers from whole-genome sequencing (WGS) data by utilizing the complete compendium of mutational signatures associated with homologous recombination deficiency. Specifically, HRDetect uses HRD-associated substitution signatures SBS3 and SBS8, HRD-associated rearrangement signatures RS3 and RS5, and indels at microhomologies reflected by HRD-associated indel signatures ID6 and ID8. CHORD is an alternative WGS-based HRD prediction tool that uses mutational patterns directly observed in cancer genomes. CHORD has similar performance to HRDetect and it is computationally efficient because it does not require derivation of mutational signatures from the observed mutational patterns. Both CHORD and HRDetect outperform SigMA. They may serve as better alternatives to conventional screening methods because they leverage all phenotypic footprints of deficiency, independent of the mechanism causing the deficiency. Further, CHORD and HRDetect capture about 50% more responders to PARP inhibitors when compared to companion diagnostic (CDx) tests. However, CHORD and HRDetect have not been widely used because they both require whole-genome sequencing data, which is generally unavailable in most clinical settings. Notably, CHORD cannot be applied to whole-exome sequenced (WES) cancers and HRDetect's performance on WES data is comparable to random guessing.

In recent years, whole-exome sequencing of cancers has become more common with multiple cancer centers and external providers routinely generating WES data for clinical decision making. Accordingly, new approaches applicable to whole-exome sequencing data are needed with improved accuracy and sensitivity.

In view of the forgoing, one objective of the present disclosure is to provide highly accurate and sensitive artificial intelligence approaches for detecting homologous recombination deficiency applicable to both whole-exome and whole-genome sequencing data.

### SUMMARY

The present technology relates to methods, systems, and devices for detecting homologous recombination deficiency in cancer. Accordingly, it is one object of the present invention to provide methods of generating a homologous recombination feature set. It is another object of the present invention to provide methods of training a predictive model configured to predict a presence of homologous recombination deficiency in a subject. It is another object of the present invention to provide methods of administering a cancer therapeutic to a subject. It is yet another object of the present invention to provide computer systems configured to output a homologous recombination classification of a subject.

In some aspects, provided are methods of generating a homologous recombination feature set. The methods include: (a) receiving a subject's sequencing data and corresponding homologous recombination classifications; and (b) generating a homologous recombination feature set, wherein the homologous recombination feature set comprises a plurality of genomic features of the subject's sequencing data and corresponding homologous recombination classifications.

In other aspects, provided are methods of training a predictive model configured to predict a presence of homologous recombination deficiency in a subject. The methods include: (a) receiving the subject's sequencing data and corresponding homologous recombination classifications; (b) generating a homologous recombination feature set, wherein the homologous recombination feature set comprises a plurality of genomic features of the subject's sequencing data and the corresponding homologous recombination classifications; and (c) training the predictive model with the homologous recombination feature set, thereby generating a trained predictive model configured to predict the presence of homologous recombination deficiency in the subject.

In other aspects, provided are methods of administering a cancer therapeutic to a subject. The methods include: (a) receiving the subject's sequencing data; (b) determining the subject's homologous recombination classification as an output of a trained predictive model, wherein the trained predictive model is provided with the subject's sequencing data as an input, and wherein the trained predictive model is trained with a homologous recombination feature set; and (c) administering the cancer therapeutic to the subject at least according to the subject's homologous recombination classification.

In further aspects, provided are a computer system configured to output a homologous recombination classification of a subject. The computer system includes: (a) one or more processors; (b) non-transient computer readable storage medium including software, wherein the software comprises executable instructions that, as a result of execution, cause the one or more processors of the computer system to: (i) receive the subject's sequencing data; and (ii) output the subject's homologous recombination classification as an output of a trained predictive model when the trained predictive model is provided with the subject's sequencing data as an input, wherein the trained predictive model is trained with a homologous recombination feature set.

The foregoing paragraphs have been provided by way of general introduction, and are not intended to limit the scope of the following claims. The described embodiments, together with further advantages, will be best understood by reference to the following detailed description taken in conjunction with the accompanying examples.

### BRIEF DESCRIPTION OF THE DRAWINGS

This application contains at least one drawing executed in color. Copies of this application with color drawing(s) will be provided by the Office upon request and payment of the necessary fees.

FIG. 1a(i)-(iii), 1b(i)-(iii), and 1c-e illustrate feature engineering to identify significantly enriched genomic components across HRD and HRP samples at both WGS and WES resolution. FIG. 1a(i)-(iii), and 1b(i)-(iii) are volcano plots with Log2 fold change (FC) enrichment across the average proportion of 96 mutation, 83 indel, and 48 copy number channels between HRD and HRP samples for 311 Sanger-WGS-Breast (1a(i)-(iii)) and 671 TCGA-WES-Breast samples (1b(i)-(iii)). Channels with an absolute FC greater than 0.5 for WGS and 0.25 for WES, and a −log₁₀ FDR adjusted p-value greater than 3 are colored. Channels colored in red are enriched in HRD samples, while channels highlighted in blue are enriched in HRP samples. FIGS. 1c-d show Principal Component Analysis (PCA) highlighting the relevance of the features derived from the significant channels in (FIG. 1a(i)-(iii), 1b(i)-(iii),) separating HRD from HRP samples across whole-genome (FIG. 1c) and whole-exome sequencing data (FIG. 1d). FIG. 1e shows feature robustness across different definitions of HRD definitions that include: (i) genomic changes in BRCA1, and BRCA2; (ii) HRD score ≥33; (iii) HRD score ≥42; (iv) HRD score ≥63; (v) presence of copy number signature CN17 associated with the HRD genomic phenotype; (vi) presence of the HRD-associated mutational signature SBS3; and (vii) HRD predictions based on SigMA. The color of the dots represents the Logz fold-change in enrichment of the six features across the HRD and HRP samples. The significance of the fold-change was calculated using Fisher's exact tests and only FDR adjusted p-values ≤0.05 are shown.

FIGS. 2a-g illustrate training HRD models for WGS and WES breast samples. FIG. 2a is a scheme that outlines the workflow for training, testing, and validating a support vector machine model for detecting HRD from WGS breast cancers. FIG. 2b shows average 10-fold cross validation weights of the six features derived from the training dataset comprised of 311 breast whole genome samples with 121 HRD and 190 HRP samples. FIG. 2c shows the average performance of the WGS HRD model on 100 random test datasets. The model achieved an AUC of 0.97 based on the receiver operating characteristic curve (ROC) and an F1 score of 0.86 based on the precision recall curve (PR). The error bars across the different performance metrics represent the standard deviation based on 100 random test datasets. FIG. 2d is a scheme that outlines the workflow for training, testing, and validating a support vector machine model for detecting HRD from WES breast cancers. FIG. 2e shows the average 10-fold cross validation weights of the six features derived from the training dataset comprised of 671 breast exome samples with 157 HRD and 514 HRP tumors. FIG. 2f shows the performance of WGS and WES HRD models of a held-out test dataset encompassing 65 samples profiled using HRDetect, SigMA, and HRProfiler. FIG. 2g shows an external validation of the HRProfiler WES model using 109 MSK-IMPACT breast cancers and a comparison with the performance of SigMA on these data.

FIGS. 3a, 3b(i)-(iv), 3c, and 3d(i)-(iii) illustrate validation and performance of HRD predictive models on WGS and downsampled WGS breast cancers. FIG. 3a shows model validation of different approaches for detecting HRD from whole-genome sequencing data based on 237 Triple Negative Breast Cancer (TNBC) samples all treated with platinum therapy. The HRProfiler model is assessed using multiple metrics and its performance is compared with the performance of SigMA, CHORD, and HRDetect. FIG. 3b(i)-(iv) show comparison of the predictive significance across HRProfiler, HRDetect, SigMA, and CHORD based on the Interval Disease Free Survival (IDFS) for 237 TNBC patients that were treated with platinum therapy. FIG. 3 c shows model performance and comparison for 237 TNBC samples downsampled to exome resolution. FIG. 3d(i)-(iii) show comparison of the predictive significance across HRProfiler, HRDetect, and SigMA based on IDFS for the down-sampled 237 TNBC samples. CHORD is not included as it cannot be applied to exome sequencing data.

FIGS. 4a-d, and 4e(i)-(iii) illustrate training and validating an HRD model for WES ovarian cancers. FIG. 4a is a scheme that outlines the workflow for training, testing, and validating a support vector machine model for detecting HRD from WES ovarian cancers. FIG. 4b shows the average 10-fold cross validation weights of the six features derived from the training dataset comprised of 182 ovarian exome samples with 82 HRD and 100 HRP samples. FIG. 4c shows the HRD model average performance based on a test dataset comprised of 41 samples. The error bars across the different performance metrics represent the standard deviation based on 100 random test datasets. The model achieved an AUC of 0.93 based on the receiver operating characteristic curve (ROC) and an F1 score of 0.78 based on the precision recall curve (PR). FIG. 4d shows model validation using 50 external MSK-IMPACT ovarian samples and performance comparison with SigMA. FIG. 4e(i)-(iii) show progression Free Interval (PFI) analysis for HRD patients stratified based on HRProfiler (q-value=0.0156; Cox proportional hazards ratio), SigMA (q-value=1; Cox proportional hazards ratio), and BRCA1/2 mutation status (q-value=1; Cox proportional hazards ratio) after correcting for age, clinical stage, and HRD score.

FIG. 5a(i)-(ii) and 5b(i)-(ii) illustrate composition of HRD and HRP samples across WGS and WES breast cancers and their associations with genomic features. FIG. 5a(i)-(ii) show distribution of HRD scores across HR pathway mutant (colored red) and WT samples (colored blue) in a subset of Sanger-WGS-breast samples and TCGA-WES-Breast samples. The table outlines the number of HRD and HRP samples across different definitions of HRD. Asterisks represent the definition used for classifying samples as HRD for all analysis in the paper across both WGS and WES samples. FIG. 5b(i)-(ii) show comparison of the proportion of APOBEC mutational signatures, SBS2 and SBS13, theacross Sanger-WGS-breast and TCGA-WES-Breast cohorts for HRD and HRP samples.

FIG. 6 is a schematic illustration of an example embodiment of a device in accordance with the present technology.

FIG. 7 is a flow diagram illustrating an example method of generating a homologous recombination feature set in accordance with the present technology.

FIG. 8 is a flow diagram illustrating an example method of training a predictive model configured to predict a presence of homologous recombination deficiency in a subject in accordance with the present technology.

FIG. 9 is a flow diagram illustrating an example method of administering a cancer therapeutic to a subject in accordance with the present technology.

### DETAILED DESCRIPTION

While the present disclosure is capable of being embodied in various forms, the description below of several embodiments is made with the understanding that the present disclosure is to be considered as an exemplification of the invention and is not intended to limit the invention to the specific embodiments illustrated. Headings are provided for convenience only and are not to be construed to limit the invention in any manner. Embodiments illustrated under any heading may be combined with embodiments illustrated under any other heading.

The terms “comprise(s)”, “include(s)”, “having”, “has”, “contain(s)”, and variants thereof, as used herein, are intended to be open-ended transitional phrases, terms, or words that do not preclude the possibility of additional acts or structures. The present disclosure also contemplates other embodiments “comprising”, “consisting of” and “consisting essentially of”, the embodiments or elements presented herein, whether explicitly set forth or not.

As used herein, the words “a” and “an” and the like carry the meaning of “one or more.”

As used herein, the word “about” may be used when describing magnitude to indicate that the value described is within a reasonable expected range of values. For example, a numeric value may have a value that is +/−0.1% of the stated value (or range of values), +/−1% of the stated value (or range of values), +/−2% of the stated value (or range of values), +/−5% of the stated value (or range of values), or +/−10% of the stated value (or range of values).

The use of numerical values in the various quantitative values specified in this application, unless expressly indicated otherwise, are stated as approximations as though the minimum and maximum values within the stated ranges were both preceded by the word “about.” It is to be understood, although not always explicitly stated, that all numerical designations are preceded by the term “about.” It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios, such as about 2, about 3, and about 4, and sub-ranges, such as about 10 to about 50, about 20 to about 100, and so forth. It also is to be understood, although not always explicitly stated, that the reagents described herein are merely exemplary and that equivalents of such are known in the art.

Where a numerical limit or range is stated herein, the endpoints are included. Also, all values and subranges within a numerical limit or range are specifically included as if explicitly written out.

The term “subject” and “patient” are used interchangeably. As used herein, they refer to any subject for whom or which therapeutic methods, including with the methods according to the present disclosure is desired. In most embodiments, the subject is a mammal, including but not limited to a human, a non-human primate such as a chimpanzee, a domestic livestock such as a cattle, a horse, a swine, a pet animal such as a dog, a cat, and a rabbit, and a laboratory subject such as a rodent, e.g., a rat, a mouse, and a guinea pig. In preferred embodiments, the subject is a human.

Repair of DNA double strand breaks by homologous recombination (HR) is an essential cellular mechanism for maintaining genomic stability and for preventing tumorigenesis¹. Prior studies have elucidated key genes in the HR pathway, including, BRCA1, BRCA2, RAD51, and PALB2, that commonly exhibit pathogenic germline variants and/or somatic mutations in breast, ovarian, prostate, and pancreatic cancers²⁻⁵. Defects in HR genes can disable the HR repair pathway making cells vulnerable to double strand breaks and, thus, providing a treatment opportunity. Specifically, patients with cancers harboring defective HR repair are highly sensitive to both poly (ADP-ribose) polymerase (PARP) inhibitors and to platinum therapies^{6,7}. PARP inhibitors induce double strand breaks by stalling the replication fork during DNA replication, thereby increasing the reliance on error-prone alternative repair pathways in HR deficient (HRD) cells, causing the cell to accumulate mutations and to consequently undergo apoptosis⁸. Similarly, platinum therapies cause inter-strand breaks, leading to p53-initiated apoptosis in HRD cells⁹.

Conventional stratification of HRD cancers and HR proficient (HRP) cancers involves screening for canonical genomic markers, including pathogenic germline variants and somatic copy number alterations in HR genes¹⁰⁻¹². Currently, there are multiple Clinical Laboratory Improvement Amendments (CLIA) certified tests and at least two U.S. Food and Drug Administration (FDA) approved commercial HRD companion diagnostic (CDx) tests available to cancer patients¹³. The FDA approved tests include Myriad myChoice® CDx and FoundationOne® CDx, which determine HRD by quantifying overall genomic instability in combination with BRCA1 and BRCA2 status¹³. For example, Myriad myChoice® CDx relies on the use of a genomic instability score (GIS) or HRD score, which is a composite score of three particular copy number alterations, including telomeric allelic imbalances (TAIs)¹², long state transition (LST) events¹⁴, and loss of heterozygosity (LOH)¹⁰. Traditionally, an HRD score cutoff of 42 has been applied to differentiate between HRD and HRP samples in metastatic breast cancers¹¹. HRD score cutoffs of 33 and 63 have been applied for ovarian cancers^{15,16}.

At least three research approaches have also been developed to capture HR deficient cancers by applying machine learning algorithms to the patterns of somatic mutations found in cancer genomes: HRDetect¹⁷, CHORD¹⁸, and SigMA¹⁹. HRDetect is a machine learning tool that detects HR deficient cancers from whole-genome sequencing (WGS) data by utilizing a subset of mutational signatures associated with homologous recombination deficiency¹⁷. Specifically, HRDetect makes use of HRD-associated single base substitution (SBS) signatures²⁰ SBS3 and SBS8, HRD-associated rearrangement signatures²¹ RS3 and RS5, and indels at microhomologies reflected by HRD-associated indel signatures²² ID6 and ID8.

CHORD is an alternative WGS-based HRD prediction tool that does not rely on mutational signatures, but it rather uses 145 types of mutations directly observed in whole-genome sequenced cancers¹⁸. CHORD is more computationally efficient and prior studies have shown that it has identical performance to the one of HRDetect¹⁷. Both CHORD and HRDetect can serve as better alternatives to conventional screening methods as they leverage phenotypic mutational footprints of deficiency, independent of the mechanism causing the deficiency^{17,18}. Further, prior studies have shown that predictions from these tools outperform conventional stratification of HRD patients²³. However, both CHORD and HRDetect rely on the use of HRD-specific patterns of structural variations that can be only reliably detected from WGS data^{17,18}. By excluding structural variations, HRDetect can also be applied to whole-exome sequencing (WES) data, albeit, with significantly diminished performance¹⁷. Conversely, CHORD's implementation does not allow utilizing WES cancers. Both CHORD and HRDetect have had only limited clinical utilization as they require whole-genome sequencing data, which is generally unavailable in most clinical settings.

In contrast to CHORD and HRDetect, SigMA was developed to detect HRD from whole-genome, whole-exome, and targeted panel sequencing data with SigMA's main focus being on panel sequencing data¹⁹. The tool utilizes a machine learning approach for exclusively identifying SBS3, but it requires a total of at least five single-base mutations from panel sequencing¹⁹. Based on MSK-IMPACT data²⁴, this limits SigMA's applicability to 35% of breast and 33% of ovarian samples as these panel sequenced samples have at least five mutations.

In principle, two distinct approaches have been utilized to evaluate the performance of methods for detecting HRD. In their original publications, CHORD and HRDetect have relied on concordance between their predictions and prior HRD/HRP annotations based on germline or somatic genomic alterations in HR pathway genes including BRCA1 and BRCA2^{17,18}. This concordance can be quantified by area under the curve of the receiver operating characteristic (AUC) with both CHORD and HRDetect reporting AUCs above 0.90^{17,18}. Unfortunately, this type of comparison requires a ground truth for HRD and HRP cancers which, in most cases, is not straightforward to derive. The second approach relies on comparing clinical endpoints of HRD-predicted and HRP-predicted cancers including overall, progression-free, and/or disease-free survival for patients treated either with platinum therapy or with PARP inhibitors. The advantage of this approach is that it provides immediate clinical relevance. Unfortunately, such comparisons require the availability of well annotated clinico-genomics datasets which are currently limited especially at the whole-genome resolution.

In recent years, whole-exome sequencing has started being integrated within clinical oncology workflows²⁵ however, there has been a lack of approaches for detecting HRD samples from exome sequenced cancers. Here, we present a highly accurate and sensitive artificial intelligence approach, termed, Homologous Recombination Proficiency Profiler (HRProfiler), for distinguishing between homologous recombination proficient (HRP) and homologous recombination deficient (HRD) breast and ovarian cancers. HRProfiler utilizes six distinct types of somatic mutations detectable from whole-exome and whole-genome sequencing data. Based on concordance between tool predictions and prior HRD/HRP annotations, HRProfiler delivers the same performance as CHORD, HRDetect, and SigMA on whole-genome sequencing data and outperforms these tools on whole-exome sequencing data. Based on clinical endpoints, HRProfiler outperforms all existing approaches in detecting patients responding to platinum therapy. Overall, HRProfiler allows using whole-exome derived mutational footprints of failed DNA repair processes for detecting clinical biomarkers for the reliable stratification of patients sensitive to PARP inhibitors or platinum therapies.

### Example Methods of Generating Homologous Recombination Deficient (HRD) Positive and HRD Negative Feature Set

In some aspects, the present disclosure provides a method of generating a homologous recombination feature set. The methods include: (a) receiving a subject's sequencing data and corresponding homologous recombination classifications; and (b) generating a homologous recombination feature set, wherein the homologous recombination feature set comprises a plurality of genomic features of the subject's sequencing data and corresponding homologous recombination classifications.

In some embodiments, the sequencing data comprises whole-genome sequencing data, whole-exome sequencing data, a fraction thereof, or any combination thereof. In some embodiments, the sequencing data comprises both whole-genome and whole-exome sequencing data.

In some embodiments, the homologous recombination feature set comprises: a total number and a proportion of deletions at microhomologies features of the sequencing data, a total number and a proportion of genomic segments with loss of heterozygosity features of the sequencing data, a total number and a proportion of heterozygous genomic segments features of the sequencing data, a total number and a proportion of C:G>T:A single base substitutions at a 5′-NpCpG-3′ contexts features of the sequencing data, a total number and a proportion of C:G>G:C single base substitutions at a 5′-NpCpT-3′ contexts features of the sequencing data, or any combination thereof.

In some embodiments, the total number and the proportions of genomic segments with loss of heterozygosity comprise a size from about 1 to about 40 megabases with at least 1 copy of the genomic segments with loss of heterozygosity. For example, the total number and the proportions of genomic segments with loss of heterozygosity comprise a size from about 1 to about 40 megabases, from about 2 to about 36 megabases, from about 4 to about 32 megabases, from about 8 to about 28 megabases, from about 12 to about 24 megabases, or from about 16 to about 20 megabases, with at least 1 copy of the genomic segments with loss of heterozygosity.

In some embodiments, the total number and the proportions of heterozygous genomic segments comprise a size from about 3 to about 40 megabases or from about 10 to about 40 megabases with 3 to 9 copies of each heterozygous genomic segment of the heterozygous genomic segments. For example, the total number and the proportions of heterozygous genomic segments comprise a size from about 10 to about 40 megabases, from about 5 to about 35 megabases, from about 7 to about 30 megabases, from about 9 to about 25 megabases, from about 11 to about 20 megabases, or from about 13 to about 15 megabases, with 3 to 9 copies, 4 to 7 copies, or 5 to 6 copies of each heterozygous genomic segment of the heterozygous genomic segments.

In other embodiments, the total number and the proportions of heterozygous genomic segments comprise a size of at least 40 megabases with 2 to 4 copies of each heterozygous genomic segment of the heterozygous genomic segments. For example, the total number and the proportions of heterozygous genomic segments comprise a size of at least 40 megabases, at least 50 megabases, at least 75 megabases, or at least 100 megabases, with 2 to 4 copies, or 3 copies of each heterozygous genomic segment of the heterozygous genomic segments.

In some embodiments, the total number and the proportions of deletions at microhomologies comprise a size of at least 5 base-pairs. For example, the total number and the proportions of deletions at microhomologies comprise a size of at least 5 base-pairs, at least 7 base-pairs, at least 9 base-pairs, at least 15 base-pairs, or at least 17 base-pairs.

In some embodiments, the homologous recombination classification comprises: homologous recombination deficiency positive or homologous recombination deficiency negative.

In some embodiments, the subject's sequencing data comprises retrospective clinical trial sequencing data of a patient that participated in a clinical trial, wherein the patient is the same as or different from the subject.

### Example Methods of Training a Homologous Recombination Deficiency Predictive Model

In other aspects, the present disclosure provides a method of training a predictive model configured to predict a presence of homologous recombination deficiency in a subject. The method includes: (a) receiving the subject's sequencing data and corresponding homologous recombination classifications; (b) generating a homologous recombination feature set, wherein the homologous recombination feature set comprises a plurality of genomic features of the subject's sequencing data and the corresponding homologous recombination classifications; and (c) training the predictive model with the homologous recombination feature set, thereby generating a trained predictive model configured to predict the presence of homologous recombination deficiency in the subject.

In some embodiments, the training includes a linear kernel support vector machine (SVM) with L1 regularization.

In some embodiments, the predictive model comprises a random forest predictive model, a naïve Bayes classifier predictive model, a support vector machine predictive model, a logistic regression predictive model, or any combination thereof.

In some embodiments, the predictive model is configured to predict the presence of homologous recombination deficiency in the subject with an accuracy of at least 60%, at least 65%, at least 70%, at least 75%, at least 80%, at least 85%, at least 90%, at least 95%, or at least 99%. For example, the accuracy may range from about 60% to about 100%, about 65% to about 99%, about 70% to about 95%, about 75% to about 90%, about 80% to about 85%.

In some embodiments, the predictive model is configured to predict the presence of homologous recombination deficiency in the subject with a precision of at least 60%, at least 65%, at least 70%, at least 75%, at least 80%, at least 85%, at least 90%, at least 95%, or at least 99%. For example, the precision may range from about 60% to about 100%, about 65% to about 99%, about 70% to about 95%, about 75% to about 90%, about 80% to about 85%.

In some embodiments, the predictive model is configured to predict the presence of homologous recombination deficiency in the subject with a F1 of at least 60%, at least 65%, at least 70%, at least 75%, at least 80%, at least 85%, at least 90%, at least 95%, or at least 99%. For example, the F1 may range from about 60% to about 100%, about 65% to about 99%, about 70% to about 95%, about 75% to about 90%, about 80% to about 85%.

In some embodiments, the predictive model is configured to predict the presence of homologous recombination deficiency in the patient with a sensitivity of at least 60%, at least 65%, at least 70%, at least 75%, at least 80%, at least 85%, at least 90%, at least 95%, or at least 99%. For example, the sensitivity may range from about 60% to about 100%, about 65% to about 99%, about 70% to about 95%, about 75% to about 90%, about 80% to about 85%.

In some embodiments, the predictive model is configured to predict the presence of homologous recombination deficiency in the patient with a specificity of at least 60%, at least 65%, at least 70%, at least 75%, at least 80%, at least 85%, at least 90%, at least 95%, or at least 99%. For example, the specificity may range from about 60% to about 100%, about 65% to about 99%, about 70% to about 95%, about 75% to about 90%, about 80% to about 85% In some embodiments, the predictive model is configured to predict the presence of homologous recombination deficiency in the patient with a balanced accuracy of at least 60%, at least 65%, at least 70%, at least 75%, at least 80%, at least 85%, at least 90%, at least 95%, or at least 99%. For example, the balanced accuracy may range from about 60% to about 100%, about 65% to about 99%, about 70% to about 95%, about 75% to about 90%, about 80% to about 85%.

In some embodiments, the sequencing data comprises whole-genome sequencing data, whole-exome sequencing data, a fraction thereof, or any combination thereof.

In some embodiments, the homologous recombination feature set comprises: a total number and proportions of deletions at microhomologies features of the sequencing data, a total number and proportions of genomic segments with loss of heterozygosity features of the sequencing data, a total number and proportions of heterozygous genomic segments features of the sequencing data, a total number and proportions of C:G>T:A single base substitutions at a 5′-NpCpG-3′ contexts features of the sequencing data, a total number and a proportion of C:G>G:C single base substitutions at a 5′-NpCpT-3′ contexts features of the sequencing data, or any combination thereof.

In some embodiments, the total number and the proportions of genomic segments with loss of heterozygosity comprise a size from about 1 to about 40 megabases with at least 1 copy of the genomic segments with loss of heterozygosity. For example, the total number and the proportions of genomic segments with loss of heterozygosity comprise a size from about 1 to about 40 megabases, from about 2 to about 36 megabases, from about 4 to about 32 megabases, from about 8 to about 28 megabases, from about 12 to about 24 megabases, or from about 16 to about 20 megabases, with at least 1 copy of the genomic segments with loss of heterozygosity.

In some embodiments, the total number and the proportions of heterozygous genomic segments comprise a size from about 3 to about 40 megabases or from about 10 to about 40 megabases with 3 to 9 copies of each heterozygous genomic segment of the heterozygous genomic segments. For example, the total number and the proportions of heterozygous genomic segments comprise a size from about 10 to about 40 megabases, from about 5 to about 35 megabases, from about 7 to about 30 megabases, from about 9 to about 25 megabases, from about 11 to about 20 megabases, or from about 13 to about 15megabases, with 3 to 9 copies, 4 to 7 copies, or 5 to 6 copies of each heterozygous genomic segment of the heterozygous genomic segments.

In other embodiments, the total number and the proportions of heterozygous genomic segments comprise a size of at least 40 megabases with 2 to 4 copies of each heterozygous genomic segment of the heterozygous genomic segments. For example, the total number and the proportions of heterozygous genomic segments comprise a size of at least 40 megabases, at least 50 megabases, at least 75 megabases, or at least 100 megabases, with 2 to 4 copies, or 3 copies of each heterozygous genomic segment of the heterozygous genomic segments.

In some embodiments, the total number and the proportions of deletions at microhomologies comprise a size of at least 5 base-pairs. For example, the total number and the proportions of deletions at microhomologies comprise a size of at least 5 base-pairs, at least 7 base-pairs, at least 9 base-pairs, at least 15 base-pairs, or at least 17 base-pairs.

In some embodiments, the homologous recombination classification comprises: homologous recombination deficiency positive or homologous recombination deficiency negative.

In some embodiments, the subject's sequencing data comprises retrospective clinical trial sequencing data of a patient that participated in a clinical trial, wherein the patient is the same as or different from the subject.

### Example Methods of Stratifying Cancer Therapeutic Administration Based on HRD Classification

In other aspects, the present disclosure provides a method of administering a cancer therapeutic to a subject. The method includes: (a) receiving the subject's sequencing data; (b) determining the subject's homologous recombination classification as an output of a trained predictive model, wherein the trained predictive model is provided with the subject's sequencing data as an input, and wherein the trained predictive model is trained with a homologous recombination feature set; and (c) administering the cancer therapeutic to the subject at least according to the subject's homologous recombination classification.

In some embodiments, the cancer therapeutic comprises at least one selected from the group consisting of platinum therapies and poly (ADP-ribose) polymerase (PARP) inhibitors. Examples of PARP inhibitors include, but are not limited to, Veliparib, Pamiparib, Talazoparib, Olaparib, Niraparib, Rucaparib, Iniparib, and 3-Aminobenzamide. In some embodiments, the PARP inhibitors comprise Talazoparib, Olaparib, Niraparib, Rucaparib, or any combination thereof. Examples of platinum therapies include, but are not limited to, Cisplatin, Oxaliplatin, Carboplatin, and Nedaplatin. In some embodiments, the platinum therapies comprise Cisplatin, Oxaliplatin, Carboplatin, or any combination thereof.

In some embodiments, the cancer therapeutic causes inter-strand breaks of genomic molecules of the subject's cells, leading to p53-initiated apoptosis.

The subject may be any subject already with cancer, a subject which does not yet experience or exhibit symptoms of cancer, or a subject predisposed to cancer. In some embodiments, the subject is a person who is predisposed to cancer, e.g., a person with a family history of cancer. For example, women who have (i) certain inherited genes (e.g., mutated BRCA1 and/or mutated BRCA2), (ii) been taking estrogen alone (without progesterone) after menopause for many years (at least 5, at least 7, or at least 10), and/or (iii) been taking fertility drug clomiphene citrate, are at a higher risk of contracting breast cancer.

In some embodiments, the subject is suspected of having cancer. Examples of cancers include, but are not limited to, bone cancer, testicular cancer, gastric cancer, sarcoma, lymphoma, Hodgkin's lymphoma, leukemia, head and neck cancer, squamous cell head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, multiple myeloma, renal cell carcinoma, bladder cancer, cervical cancer, choriocarcinoma, colon cancer, oral cancer, skin cancer, and melanoma.

In some embodiments, the cancer is at least one selected from the group consisting of breast cancer, ovarian cancer, prostate cancer, pancreatic cancer, and sarcoma.

In some embodiments, the trained predictive model comprises a predictive model trained by a linear kernel support vector machine (SVM) with L1 regularization.

In some embodiments, the trained predictive model is configured to determine the subject's homologous recombination classification with an accuracy of at least 60%, at least 65%, at least 70%, at least 75%, at least 80%, at least 85%, at least 90%, at least 95%, or at least 99%. For example, the accuracy may range from about 60% to about 100%, about 65% to about 99%, about 70% to about 95%, about 75% to about 90%, about 80% to about 85%.

In some embodiments, the trained predictive model is configured to determine the subject's homologous recombination classification with a precision of at least 60%, at least 65%, at least 70%, at least 75%, at least 80%, at least 85%, at least 90%, at least 95%, or at least 99%. For example, the precision may range from about 60% to about 100%, about 65% to about 99%, about 70% to about 95%, about 75% to about 90%, about 80% to about 85%.

In some embodiments, the trained predictive model is configured to determine the subject's homologous recombination classification with a F1 of at least 60%, at least 65%, at least 70%, at least 75%, at least 80%, at least 85%, at least 90%, at least 95%, or at least 99%. For example, the F1 may range from about 60% to about 100%, about 65% to about 99%, about 70% to about 95%, about 75% to about 90%, about 80% to about 85%.

In some embodiments, the trained predictive model is configured to determine the subject's homologous recombination classification with a sensitivity of at least 60%, at least 65%, at least 70%, at least 75%, at least 80%, at least 85%, at least 90%, at least 95%, or at least 99%. For example, the sensitivity may range from about 60% to about 100%, about 65% to about 99%, about 70% to about 95%, about 75% to about 90%, about 80% to about 85%.

In some embodiments, the trained predictive model is configured to determine the subject's homologous recombination classification with a specificity of at least 60%, at least 65%, at least 70%, at least 75%, at least 80%, at least 85%, at least 90%, at least 95%, or at least 99%. For example, the specificity may range from about 60% to about 100%, about 65% to about 99%, about 70% to about 95%, about 75% to about 90%, about 80% to about 85%.

In some embodiments, the trained predictive model is configured to determine the subject's homologous recombination classification with a balanced accuracy of at least 60%, at least 65%, at least 70%, at least 75%, at least 80%, at least 85%, at least 90%, at least 95%, or at least 99%. For example, the balanced accuracy may range from about 60% to about 100%, about 65% to about 99%, about 70% to about 95%, about 75% to about 90%, about 80% to about 85%.

In some embodiments, the sequencing data comprises whole-genome sequencing data, whole-exome sequencing data, any fraction thereof, or any combination thereof. In some embodiments, the sequencing data comprises both whole-genome and whole-exome sequencing data.

In some embodiments, the homologous recombination feature set comprises genomic features.

In some embodiments, the genomic features comprise: a total number and proportions of deletions at microhomologies features of the sequencing data, a total number and proportions of genomic segments with loss of heterozygosity features of the sequencing data, a total number and proportions of heterozygous genomic segments features of the sequencing data, a total number and proportions of C:G>T:A single base substitutions at a 5′-NpCpG-3′ contexts features of the sequencing data, a total number and a proportion of C:G>G:C single base substitutions at a 5′-NpCpT-3′ contexts features of the sequencing data, or any combination thereof.

In some embodiments, the total number and the proportion of genomic segments with loss of heterozygosity comprise a size from about 1 to about 40 megabases with at least 1 copy of the genomic segments with loss of heterozygosity. For example, the total number and the proportions of genomic segments with loss of heterozygosity comprise a size from about 1 to about 40 megabases, from about 2 to about 36 megabases, from about 4 to about 32 megabases, from about 8 to about 28 megabases, from about 12 to about 24 megabases, or from about 16 to about 20 megabases, with at least 1 copy of the genomic segments with loss of heterozygosity.

In some embodiments, the total number and the proportion of heterozygous genomic segments comprise a size from about 3 to about 40 megabases or from about 10 to about 40 megabases with 3 to 9 copies of each heterozygous genomic segment of the heterozygous genomic segments. For example, the total number and the proportions of heterozygous genomic segments comprise a size from about 10 to about 40 megabases, from about 5 to about 35 megabases, from about 7 to about 30 megabases, from about 9 to about 25 megabases, from about 11 to about 20 megabases, or from about 13 to about 15 megabases, with 3 to 9 copies, 4 to 7 copies, or 5 to 6 copies of each heterozygous genomic segment of the heterozygous genomic segments.

In other embodiments, the total number and the proportion of heterozygous genomic segments comprise a size of at least 40 megabases with 2 to 4 copies of each heterozygous genomic segment of the heterozygous genomic segments. For example, the total number and the proportions of heterozygous genomic segments comprise a size of at least 40 megabases, at least 50 megabases, at least 75 megabases, or at least 100 megabases, with 2 to 4 copies, or 3 copies of each heterozygous genomic segment of the heterozygous genomic segments.

In some embodiments, the total number and the proportion of deletions at microhomologies comprise a size of at least 5 base-pairs. For example, the total number and the proportions of deletions at microhomologies comprise a size of at least 5 base-pairs, at least 7 base-pairs, at least 9 base-pairs, at least 15 base-pairs, or at least 17 base-pairs.

In some embodiments, the homologous recombination classification comprises: homologous recombination deficiency positive or homologous recombination deficiency negative.

In some embodiments, the subject's sequencing data comprises retrospective clinical trial sequencing data of a patient that participated in a clinical trial, wherein the patient is the same as or different from the subject.

### Example System Configured to Determine HRD Classification of a Subject Using a Trained HRD Model

In further aspects, the present disclosure provides a computer system configured to output a homologous recombination classification of a subject. The computer system includes: (a) one or more processors; (b) non-transient computer readable storage medium including software, wherein the software comprises executable instructions that, as a result of execution, cause the one or more processors of the computer system to: (i) receive the subject's sequencing data; and (ii) output the subject's homologous recombination classification as an output of a trained predictive model when the trained predictive model is provided with the subject's sequencing data as an input, wherein the trained predictive model is trained with a homologous recombination feature set.

In some embodiments, the software comprises determining a cancer therapeutic at least according to the subject's homologous recombination classification.

In some embodiments, the cancer therapeutic comprises at least one selected from the group consisting of platinum therapies and poly (ADP-ribose) polymerase (PARP) inhibitors. In some embodiments, the PARP inhibitors comprise Talazoparib, Olaparib, Niraparib, Rucaparib, or any combination thereof. In some embodiments, the platinum therapies comprise Cisplatin, Oxaliplatin, Carboplatin, or any combination thereof.

In some embodiments, the cancer therapeutic causes inter-strand breaks of genomic molecules of the subject's cells, leading to p53-initiated apoptosis.

The subject may be any subject already with cancer, a subject which does not yet experience or exhibit symptoms of cancer, or a subject predisposed to cancer. In some embodiments, the subject is a person who is predisposed to cancer, e.g., a person with a family history of cancer. For example, women who have (i) certain inherited genes (e.g., mutated BRCA1 and/or mutated BRCA2), (ii) been taking estrogen alone (without progesterone) after menopause for many years (at least 5, at least 7, or at least 10), and/or (iii) been taking fertility drug clomiphene citrate, are at a higher risk of contracting breast cancer.

In some embodiments, the subject is suspected of having cancer. Examples of cancers include, but are not limited to, bone cancer, testicular cancer, gastric cancer, sarcoma, lymphoma, Hodgkin's lymphoma, leukemia, head and neck cancer, squamous cell head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, multiple myeloma, renal cell carcinoma, bladder cancer, cervical cancer, choriocarcinoma, colon cancer, oral cancer, skin cancer, and melanoma.

In some embodiments, the cancer is at least one selected from the group consisting of breast cancer, ovarian cancer, prostate cancer, pancreatic cancer, and sarcoma.

In some embodiments, the trained predictive model comprises a predictive model trained by a linear kernel support vector machine (SVM) with L1 regularization.

In some embodiments, the sequencing data comprises whole-genome sequencing data, whole-exome sequencing data, a fraction thereof, or any combination thereof. In some embodiments, the sequencing data comprises both whole-genome and whole-exome sequencing data.

In some embodiments, the homologous recombination feature set comprises genomic features.

In some embodiments, the genomic features comprise: a total number and a proportion of deletions at microhomologies features of the sequencing data, a total number and a proportion of genomic segments with loss of heterozygosity features of the sequencing data, a total number and a proportion of heterozygous genomic segments features of the sequencing data, a total number and a proportion of C:G>T:A single base substitutions at a 5′-NpCpG-3′ contexts features of the sequencing data, a total number and a proportion of C:G>G:C single base substitutions at a 5′-NpCpT-3′ contexts features of the sequencing data, or any combination thereof.

In some embodiments, the total number and the proportions of genomic segments with loss of heterozygosity comprise a size from about 1 to about 40 megabases with at least 1 copy of the genomic segments with loss of heterozygosity. For example, the total number and the proportions of genomic segments with loss of heterozygosity comprise a size from about 1 to about 40 megabases, from about 2 to about 36 megabases, from about 4 to about 32 megabases, from about 8 to about 28 megabases, from about 12 to about 24 megabases, or from about 16 to about 20 megabases, with at least 1 copy of the genomic segments with loss of heterozygosity.

In some embodiments, the total number and the proportions of heterozygous genomic segments comprise a size from about 3 to about 40 megabases or from about 10 to about 40 megabases with 3 to 9 copies of each heterozygous genomic segment of the heterozygous genomic segments. For example, the total number and the proportions of heterozygous genomic segments comprise a size from about 10 to about 40 megabases, from about 5 to about 35 megabases, from about 7 to about 30 megabases, from about 9 to about 25 megabases, from about 11 to about 20 megabases, or from about 13 to about 15 megabases, with 3 to 9 copies, 4 to 7 copies, or 5 to 6 copies of each heterozygous genomic segment of the heterozygous genomic segments.

In other embodiments, the total number and the proportions of heterozygous genomic segments comprise a size of at least 40 megabases with 2 to 4 copies of each heterozygous genomic segment of the heterozygous genomic segments. For example, the total number and the proportions of heterozygous genomic segments comprise a size of at least 40 megabases, at least 50 megabases, at least 75 megabases, or at least 100 megabases, with 2 to 4 copies, or 3 copies of each heterozygous genomic segment of the heterozygous genomic segments.

In some embodiments, the total number and the proportions of deletions at microhomologies comprise a size of at least 5 base-pairs. For example, the total number and the proportions of deletions at microhomologies comprise a size of at least 5 base-pairs, at least 7 base-pairs, at least 9 base-pairs, at least 15 base-pairs, or at least 17 base-pairs.

In some embodiments, the homologous recombination classification comprises: homologous recombination deficiency positive or homologous recombination deficiency negative.

In some embodiments, the trained predictive model is configured to output the subject's homologous recombination classification with an accuracy of at least 60%, at least 65%, at least 70%, at least 75%, at least 80%, at least 85%, at least 90%, at least 95%, or at least 99%. For example, the accuracy may range from about 60% to about 100%, about 65% to about 99%, about 70% to about 95%, about 75% to about 90%, about 80% to about 85%.

In some embodiments, the trained predictive model is configured to output the subject's homologous recombination classification with a precision of at least 60%, at least 65%, at least 70%, at least 75%, at least 80%, at least 85%, at least 90%, at least 95%, or at least 99%. For example, the precision may range from about 60% to about 100%, about 65% to about 99%, about 70% to about 95%, about 75% to about 90%, about 80% to about 85%.

In some embodiments, the trained predictive model is configured to output the subject's homologous recombination classification with a F1 of at least 60%, at least 65%, at least 70%, at least 75%, at least 80%, at least 85%, at least 90%, at least 95%, or at least 99%. For example, the F1 may range from about 60% to about 100%, about 65% to about 99%, about 70% to about 95%, about 75% to about 90%, about 80% to about 85%.

In some embodiments, the trained predictive model is configured to output the subject's homologous recombination classification with a sensitivity of at least 60%, at least 65%, at least 70%, at least 75%, at least 80%, at least 85%, at least 90%, at least 95%, or at least 99%. For example, the sensitivity may range from about 60% to about 100%, about 65% to about 99%, about 70% to about 95%, about 75% to about 90%, about 80% to about 85%.

In some embodiments, the trained predictive model is configured to output the subject's homologous recombination classification with a specificity of at least 60%, at least 65%, at least 70%, at least 75%, at least 80%, at least 85%, at least 90%, at least 95%, or at least 99%. For example, the specificity may range from about 60% to about 100%, about 65% to about 99%, about 70% to about 95%, about 75% to about 90%, about 80% to about 85%.

In some embodiments, the trained predictive model is configured to output the subject's homologous recombination classification with a balanced accuracy of at least 60%, at least 65%, at least 70%, at least 75%, at least 80%, at least 85%, at least 90%, at least 95%, or at least 99%. For example, the balanced accuracy may range from about 60% to about 100%, about 65% to about 99%, about 70% to about 95%, about 75% to about 90%, about 80% to about 85%.

In some embodiments, the subject's sequencing data comprises retrospective clinical trial sequencing data of a patient that participated in a clinical trial, wherein the patient is the same as or different from the subject.

Obviously, numerous modifications and variations of the present invention are possible in light of the above teachings. It is therefore to be understood that, within the scope of the appended claims, the invention may be practiced otherwise than as specifically described herein.

The examples below are intended to further illustrate protocols for preparing, characterizing, and using the complexes of the present disclosure, and are not intended to limit the scope of the claims.

### EXAMPLES

### Example 1

### HRD Genomics Biomarker

Repair of DNA double strand breaks by homologous recombination (HR) is an essential cellular mechanism for maintaining genomic stability and preventing tumorigenesis. Prior studies have elucidated key genes in the HR pathway, including, BRCA1, BRCA2, RAD51, and PALB2, that commonly exhibit germline or somatic mutations in breast, ovarian, and pancreatic cancers. Defects in HR genes can disable the HR repair pathway making cells vulnerable to double strand breaks and, thus, providing a treatment opportunity. Specifically, patients with cancers harboring defective HR repair are highly sensitive to both poly (ADP-ribose) polymerase (PARP) inhibitors and platinum therapies. PARP inhibitors induce double strand breaks by stalling the replication fork during DNA replication, thereby increasing the reliance on error-prone alternative repair pathways in HR deficient (HRD) cells, causing the cell to accumulate mutations and to consequently undergo apoptosis. Similarly, platinum therapies cause inter-strand breaks, leading to p53-initiated apoptosis in HRD cells.

Conventional stratification of HRD patients involves screening for canonical genomic markers, including pathogenic germline variants and somatic copy number alterations in HR genes. Two commercial HRD companion diagnostic (CDx) tests have been approved by the U.S. Food and Drug Administration for patients with ovarian cancer. Myriad myChoice® CDx and FoundationOne® CDx both determine HRD by quantifying overall genomic instability in combination with BRCA1 and BRCA2 status. At least three academic approaches have also been developed to capture HR deficient cancers by applying machine learning approaches to the patterns of somatic mutations found in cancer sequencing data: SigMA, HRDetect, and CHORD. SigMA was specifically developed to detect SBS3, a mutational signature of single base substitutions (SBS) previously attributed to HRD, from targeted panel and whole-exome sequencing data. Unfortunately, SigMA is only applicable to targeted panel and whole-exome sequencing data only from highly mutated cancers (<15% of all breast, ovarian, and pancreatic cancers). HRDetect is a machine learning tool that detects HR deficient cancers from whole-genome sequencing (WGS) data by utilizing the complete compendium of mutational signatures associated with homologous recombination deficiency. Specifically, HRDetect makes use of HRD-associated substitution signatures SBS3 and SBS8, HRD-associated rearrangement signatures RS3 and RS5, and indels at microhomologies reflected by HRD-associated indel signatures ID6 and ID8. CHORD is an alternative WGS-based HRD prediction tool that uses the directly observed mutational patterns of cancer genomes. CHORD is more computationally efficient, as it does not require deriving mutational signatures from the observed mutational patterns, and it has similar performance to HRDetect. Both CHORD and HRDetect outperform SigMA and they can serve as better alternatives to conventional screening methods as they leverage all phenotypic footprints of deficiency, independent of the mechanism causing the deficiency. Further, CHORD and HRDetect capture ~50% more responders to PARP inhibitors when compared to companion diagnostic (CDx) tests. However, CHORD and HRDetect have had only limited clinical utilization as they require whole-genome sequencing data, which is generally unavailable in most clinical settings. Importantly, CHORD cannot be applied to whole-exome sequenced (WES) cancers while HRDetect's performance on WES data is comparable to random guessing. In recent years, whole-exome sequencing of cancers has become more common with multiple cancer centers and external providers routinely generating WES data for clinical decision making.

The present disclosure presents a highly accurate and sensitive artificial intelligence approach for detecting homologous recombination deficiency applicable to both whole-exome and whole-genome sequencing data. The approach disclosed herein uses a minimum set of six genomic features encompassing: (i) total number and proportion of deletions spanning at least 5 base pairs (bp) at microhomologies; (ii) total number and proportion of genomic segments with loss of heterozygosity (LOH) with sizes between 1 and 40 megabases; (iii) total number and proportion of heterozygous genomic segments with Total Copy Number (TCN) between 3 and 9 and sizes between 10 and 40 megabases; (iv) total number and proportion of heterozygous genomic segments with TCN between 2 and 4 and sizes above 40 megabases; (v) total number and proportion of C:G>T:A single base substitutions at 5′-NpCpG-3′ context (mutated based underlined; N reflects any base 5′ of the mutated cytosine); and (vi) total number and proportion of C:G>G:C single base substitutions at 5′-NpCpT-3′ context. By applying a linear kernel support vector machine (SVM) with L1 regularization to these features, we have trained an AI approach for predicting homologous recombination deficiency. The training of the model and prediction is applicable to both whole-genome and whole-exome sequencing data. The trained model outperforms SigMA, CHORD, and HRDetect on whole-genome and whole-exome sequencing data. Notably, the trained model provides the same resolution for detecting homologous recombination deficiency from whole-exome sequenced samples making it immediately applicable into a clinical setting. Overall, the developed AI approach bridges the gap in using the molecular phenotypic footprint of failed DNA repair processes as clinical biomarkers for the reliable stratification of patients sensitive to PARP inhibitors and/or platinum therapies.

### Example 2

### Generation, Training, and Application of HRD Models

A minimum set of six genomic features encompassing the following features was used: (i) total number and proportion of deletions spanning at least 5 base pairs (bp) at microhomologies; (ii) total number and proportion of genomic segments with loss of heterozygosity (LOH) with sizes between 1 and 40 megabases; (iii) total number and proportion of heterozygous genomic segments with Total Copy Number (TCN) between 3 and 9 and sizes between 10 and 40 megabases; (iv) total number and proportion of heterozygous genomic segments with TCN between 2 and 4 and sizes above 40 megabases; (v) total number and proportion of C:G>T:A single base substitutions at 5′-NpCpG-3′ context (mutated based underlined; N reflects any base 5′ of the mutated cytosine); and (vi) total number and proportion of C:G>G:C single base substitutions at 5′-NpCpT-3′ context.

By applying a linear kernel support vector machine (SVM) with L1 regularization to the above features, an AI approach for predicting homologous recombination deficiency was trained. The training of the model and prediction was applicable to both whole-genome and whole-exome sequencing data. The trained model outperformed SigMA, CHORD, and HRDetect on whole-genome and whole-exome sequencing data.

Notably, the trained model provided the same resolution for detecting homologous recombination deficiency from whole-exome sequenced samples, demonstrating that it's immediately applicable to clinical settings. Overall, the developed AI approach has succeeded in using the molecular phenotypic footprint of failed DNA repair processes as clinical biomarkers for a reliable stratification of patients sensitive to PARP inhibitors and/or platinum therapies.

The approach described herein is readily applicable to any exome sequencing data. Essentially, the invention allows detecting HRD status from these sequencing data and can be applied for identifying better treatment of multiple cancer types, including, but not limited to: breast cancer, ovarian cancer, pancreatic cancer, prostate cancer, and sarcoma. Potential commercial applications of the invention include precision oncology, e.g., identification of cancer patients who would respond to platinum and/or PARP therapies.

### Example 3

### Feature Engineering of Mutation Types Enriched in HRD Samples

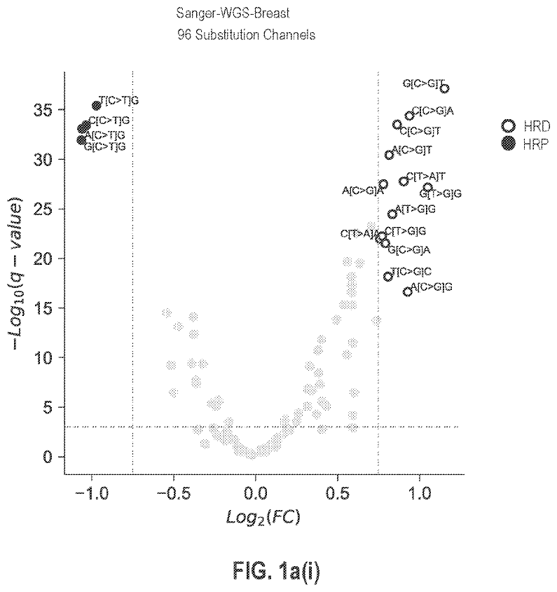

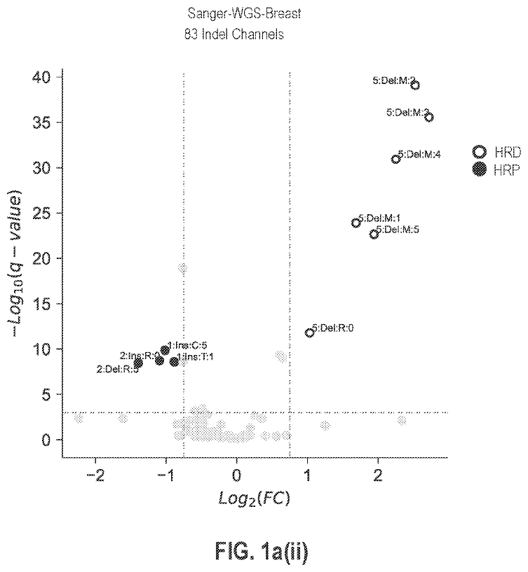

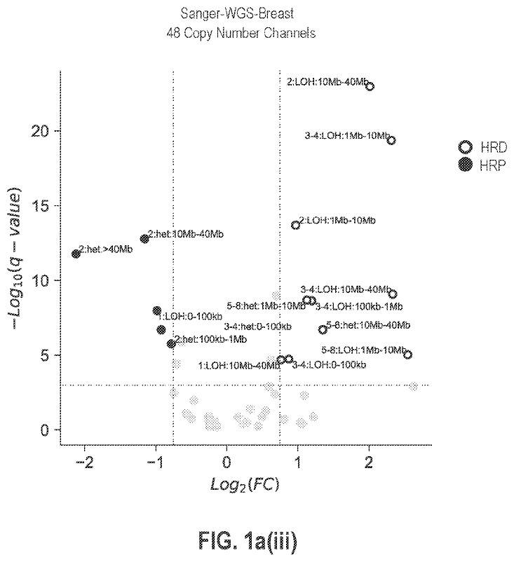

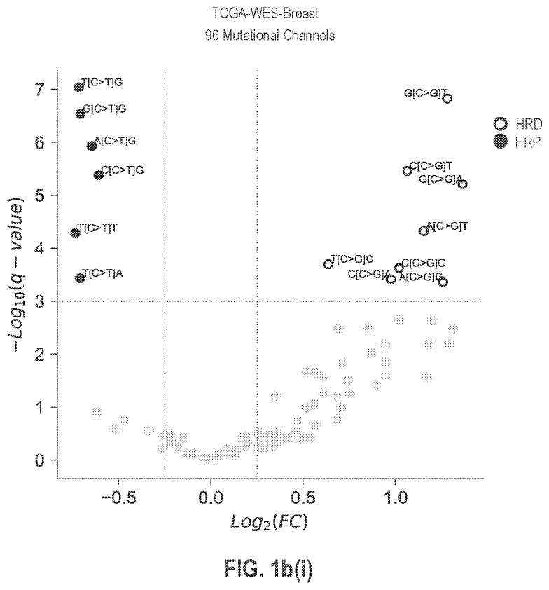

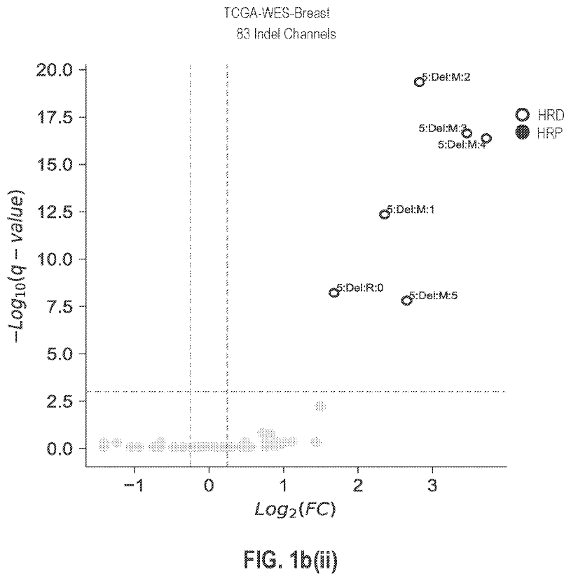

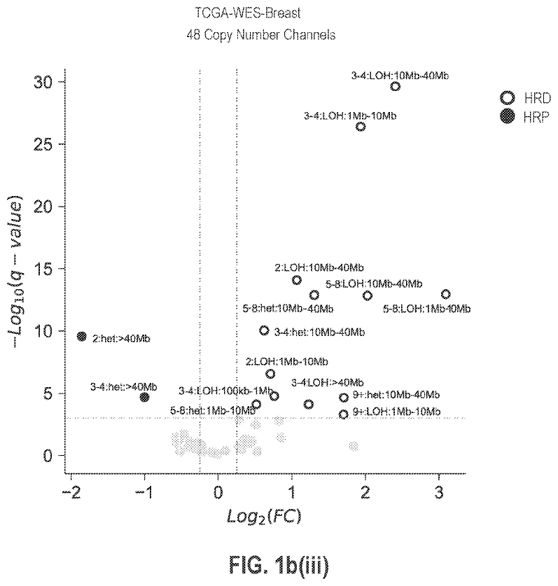

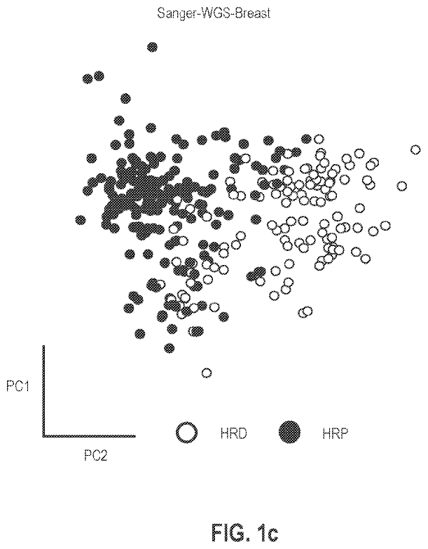

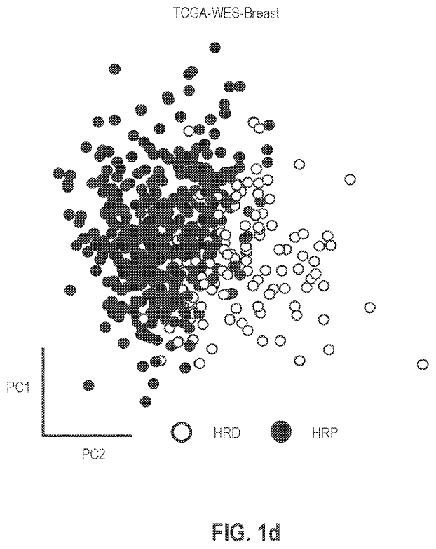

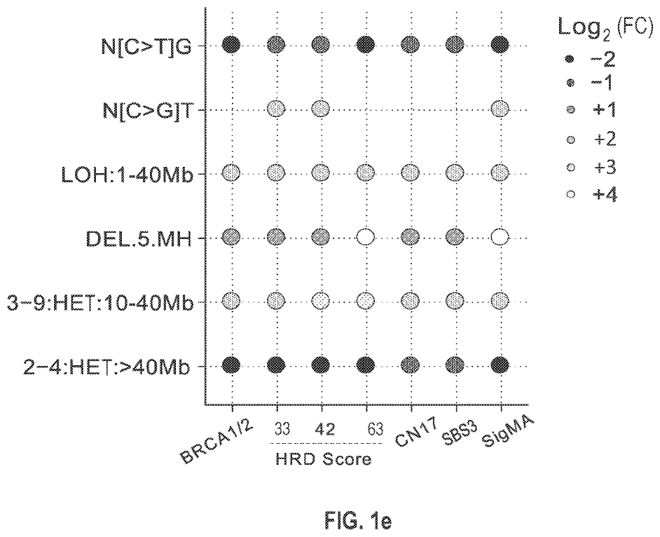

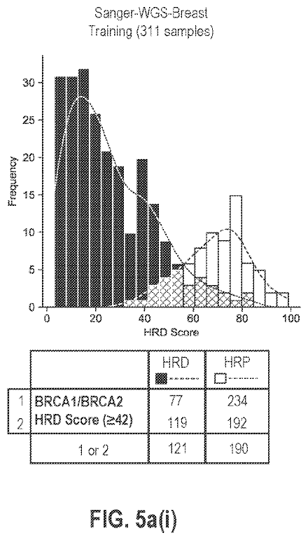

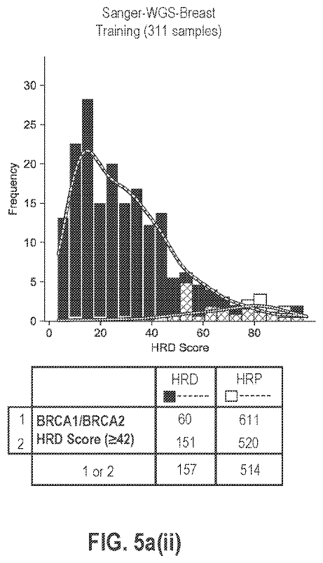

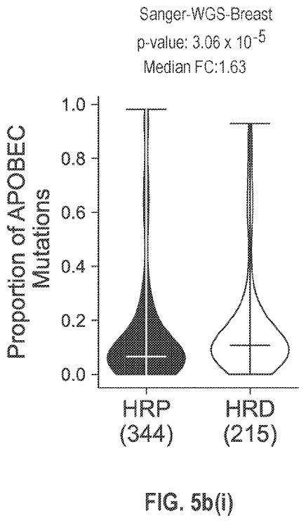

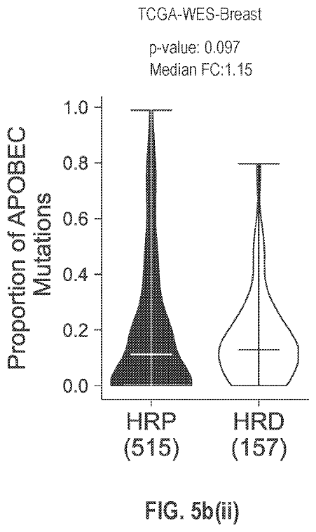

To determine the genomic footprints of homologous recombination deficiency (HRD) across patients profiled using WGS and WES, significantly enriched mutation types specific to single-base substitutions (SBSs)²⁶, insertions and deletions (IDs)²⁷, and copy number alterations (CNs)²⁸ were identified. In particular, using previously developed schemes for classifying SBSs, DBSs, and CNs^{27,29}, the types of somatic mutations enriched in either HRD cancer or in HR proficient (HRP) cancers were compared. Comparisons were performed for whole-genome sequenced breast cancers using a subset of the Sanger Institute's 560 breast cancer genomes cohort²¹ (Sanger-WGS-Breast; FIG. 1 a(i)-(iii)) as well as for whole-exome sequenced breast cancers using a subset of the TCGA breast cancer cohort³⁰ (TCGA-WES-Breast; FIG. 1b(i)-(iii)). For feature engineering and training purposes, patients were classified as HRD either based on HRD score of at least 42 and/or based on the presence of pathogenic germline variants, somatic mutations, or methylation of BRCA1 and BRCA2 (FIG. 5a(i)-(ii)).

At the SBS resolution, a striking enrichment of C:G>T:A single base substitutions were observed at 5′-NpCpG-3′ context (mutated based underlined; N reflects any base 5′ of the mutated cytosine) in HRP samples (FIG. 1a(i)-(iii) and 1b(i)-(iii)). This suggested that a relatively larger proportion of mutations in HRP samples are C:G>T:A transitions at CpG sites when compared to HRD samples. Conversely, HRD samples were enriched for C:G>G:C single base substitutions at 5′-NpCpT-3′ context. At the indel resolution, an enrichment of deletions was observed spanning at least 5 base pairs (bp) with flanking microhomology sequences across HRD samples (FIG. 1a(i)-(iii) and 1b(i)-(iii)). These mutations could arise from the erroneous activity of the Microhomology Mediated End Joining (MMEJ) or the Single Strand Annealing (SSA) DNA repair pathways in the absence of a functional HR pathway³¹. At the copy number resolution, Loss of Heterozygosity (LOH) events spanning 1 to 40Mb and heterozygous events spanning 10 to 40Mb with a Total Copy Number (TCN) state between 3 and 9 were enriched in HRD samples (FIG. 1a(i)-(iii) and 1b(i)-(iii)). On the contrary, very large (>40 Mb) heterozygous segments with TCN between 2 and 4 were enriched in HRP samples (FIG. 1a(i)-(iii) and 1b(i)-(iii)). This finding suggests that very large diploid segments or regions that have undergone genome-doubling are enriched in HRP samples, in line with the observation that HRP samples are genomically stable, harbor relatively low copy number aberrations, and thus, have a lower HRD score compared to HRD samples³².

Based on these observations, the significant mutational channels (Methods) were combined into the following six genomic features: (i) total number and proportion of deletions spanning at least 5 bp at microhomologies (abbreviated as DEL.5.MH); (ii) total number and proportion of genomic segments with loss of heterozygosity (LOH) with sizes between 1 and 40 megabases (LOH: 1-40 Mb); (iii) total number and proportion of heterozygous genomic segments with TCN between 3 and 9 and sizes between 10 and 40 megabases (3-9:HET: 10-40 Mb); (iv) total number and proportion of heterozygous genomic segments with TCN between 2 and 4 and sizes above 40 megabases (2-4: Het:>40 Mb); (v) total number and proportion of C:G>T: A single base substitutions at 5′-NpCpG-3′ context (N[C>T]G); and (vi) total number and proportion of C:G>G:C single base substitutions at 5′-NpCpT-3′ context (N[C>G]T). To determine if these genomic features can accurately separate HRD and HRP samples, a Principal Component analysis (PCA) was conducted, which showed that these six features can discern HRD from HRP samples across the two principal components for both WGS (FIG. 1c) and WES (FIG. 1d) samples.

Next, using the TCGA-WES-Breast cohort, the associations of the six genomic features were compared with previous developed HRD annotations, including: (i) germline or somatic alterations in BRCA1/2, (ii) different thresholds for HRD score previously reported in the literature^{11,15,16}, (iii) copy number HRD signature CN1729, (iv) signature SBS3 based on COSMIC attributions²⁷, and (v) signature SBS3 based on SigMA attributions¹⁹ (FIG. 1e). In all cases, the six genomic features were highly associated across majority of the HRD annotations with N[C>T]G and 2-4: HET:>40 Mb enriched in HRP samples and all other features enriched in HRD samples (FIG. 1e).

### Example 4

### Training Models to Detect HRD From WGS and WES Breast Cancer

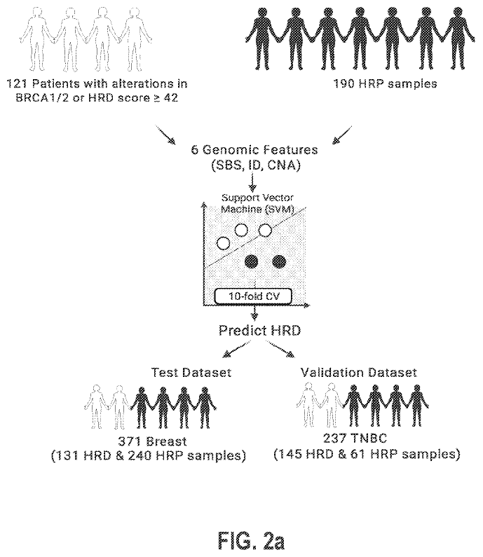

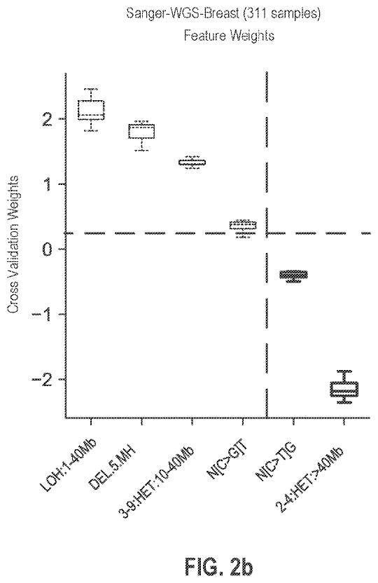

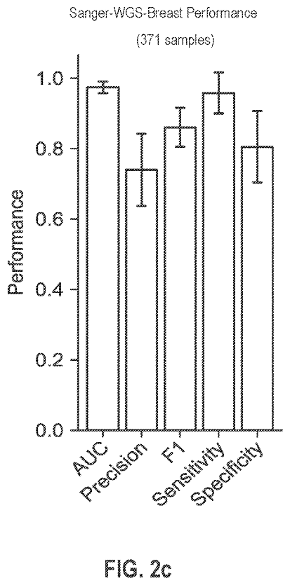

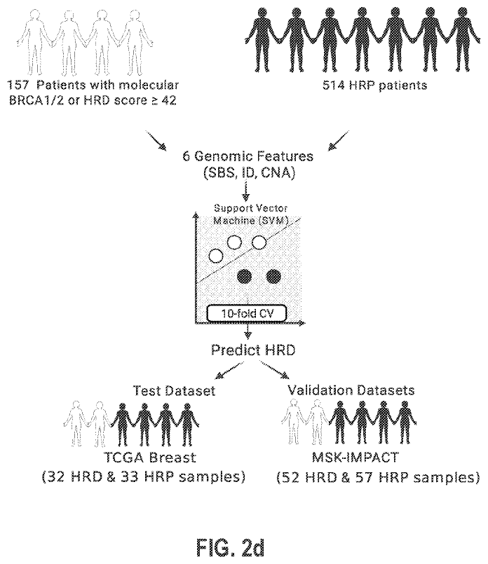

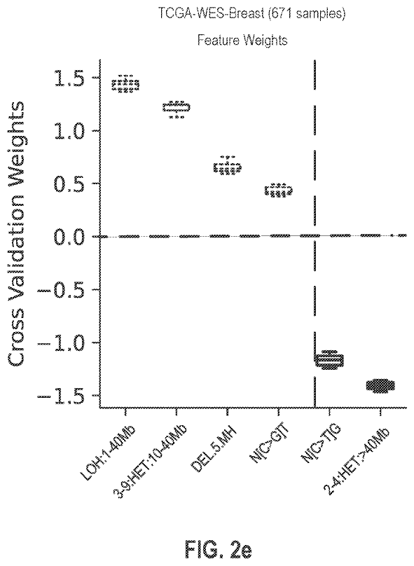

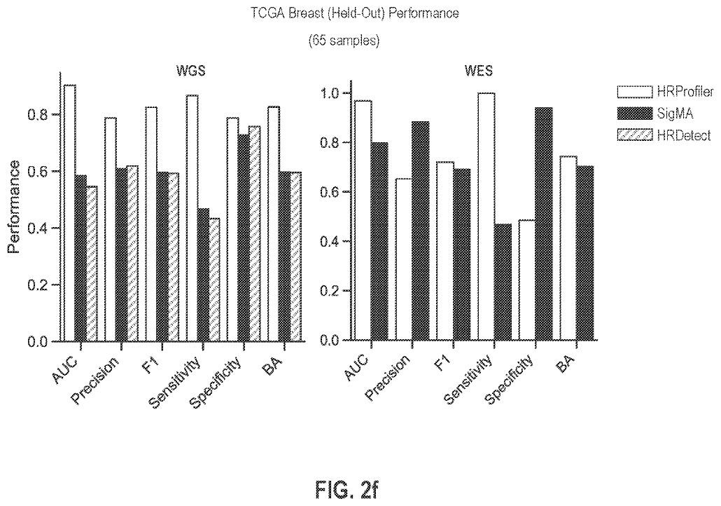

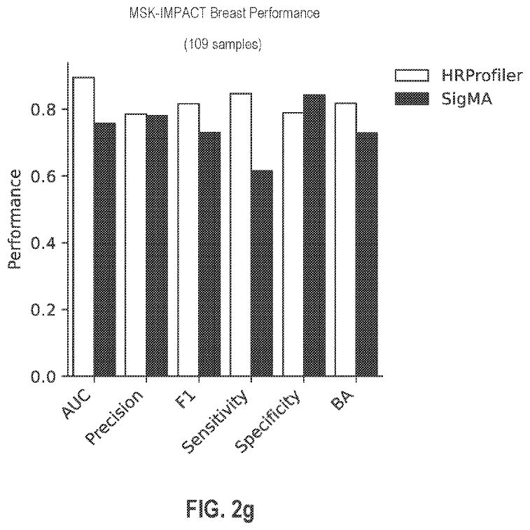

To determine if the defined genomic features can accurately predict HRD status at the WGS resolution, a machine learning model, termed, HRProfiler, was trained based on linear kernel support vector machine (SVM) using 311 samples, including, 121 HRD and 190 HRP cancers, from the Sanger-WGS-Breast dataset (FIG. 2a). For training purposes, patients were classified as HRD based on genomic alterations in BRCA1 and BRCA2 or an HRD score of at least 42. Ten-fold cross validation were conducted to determine the feature weights for the trained model (FIG. 2b). Features with positive weights (LOH: 1-40 Mb, DEL.5.MH, 3-9: HET: 10-40 Mb, and N[C>G]T) were enriched in HRD samples, whereas, features with negative weights (N[C>T]G and 2-4: Het:>40 Mb) were enriched in HRP samples. The model's performance was tested on a total of 371 samples that comprised of 311 training samples and 60 held-out HRP samples. To ensure robustness of the model's performance, the model was run across 100 random test datasets generated by randomly sampling 20% of the entire dataset. HRProfiler had an average AUC of 0.97 and an F1-score of 0.86 across the 100 test datasets, providing comparable performance to other tools on the same dataset¹⁷ (FIG. 2c). To determine the applicability of genomic features at the exome resolution for HRD prediction, a breast-specific exome HRProfiler model was further trained by applying SVM to 671 TCGA-WES-Breast cancers, comprised of 157 HRD and 514 HRP tumors (FIG. 2d). For training purposes, patients were classified as HRD based on genomic changes in BRCA1, and BRCA2 or an HRD score of at least 42. Feature importance based on ten-fold cross-validation of the HRProfiler model demonstrates the robustness of the genomic features with LOH:1-40 Mb, DEL.5.MH, and 3-9: HET: 10-40 Mb, and N[C>G]T consistently enriched in HRD and N[C>T]G and 2-4: Het:>40 Mb enriched in HRP samples (FIG. 2e). To compare the performance of HRProfiler in predicting HRD status for breast samples profiled at both whole-genome and whole-exome resolution, the HRD status was determined for 65 held-out TCGA Breast samples, profiled using both WGS and WES, by applying a whole-genome and exome-based HRProfiler model respectively (FIG. 2f). At both WGS and WES resolution, HRProfiler outperformed SigMA and HRDetect in predicting HRD status for the breast samples, thereby highlighting the generalizability of the six features in predicting HRD status for both WGS and WES samples. To further validate the performance of HRProfiler on an external independent dataset, HRD probabilities were predicted using HRProfiler for 109 exome MSK-IMPACT breast samples and a higher sensitivity, AUC and F1 score compared to SigMA were reported (FIG. 2g).

### Example 5

### Detecting HRD from WGS and Down-Sampled WGS Breast Samples

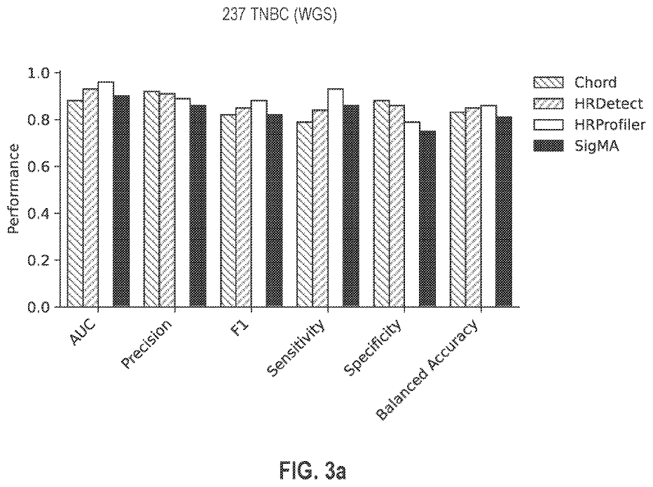

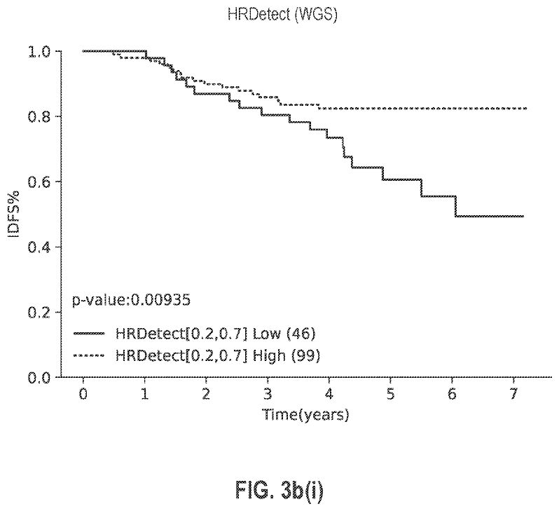

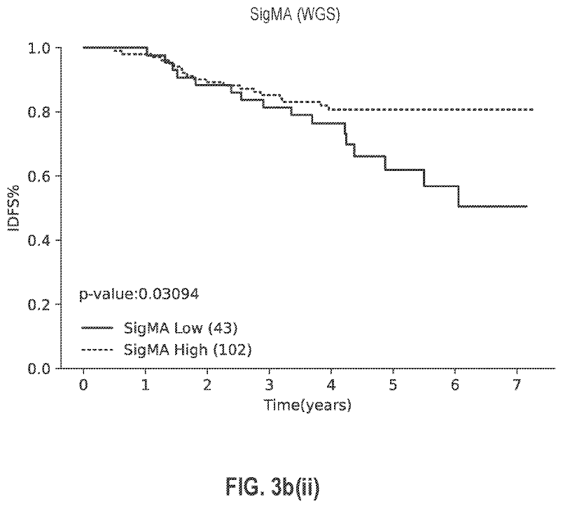

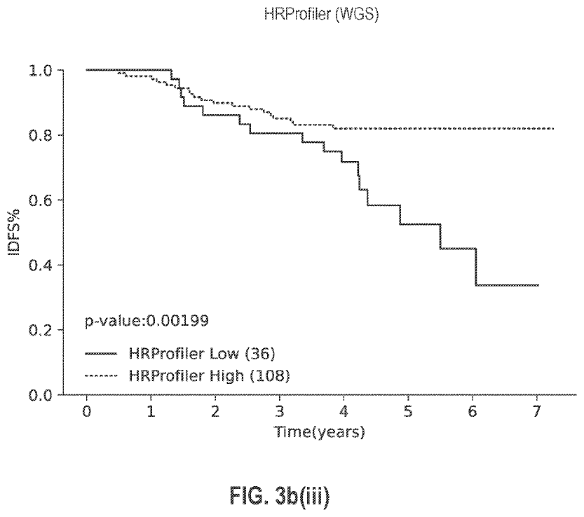

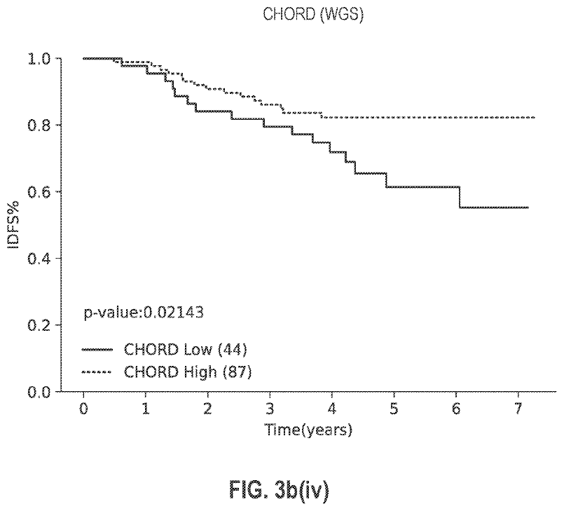

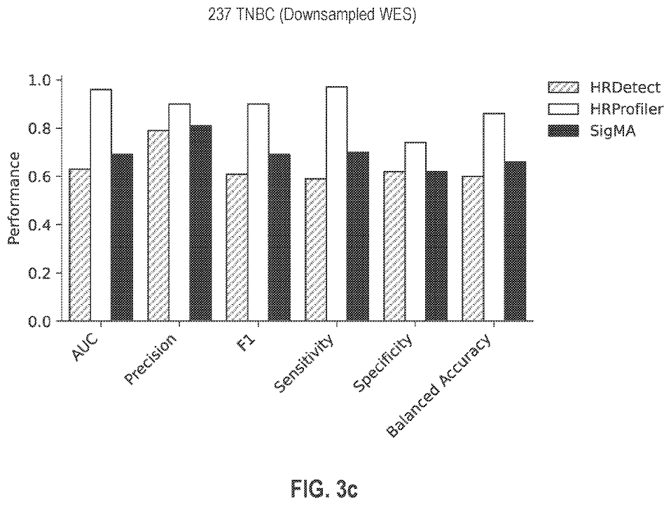

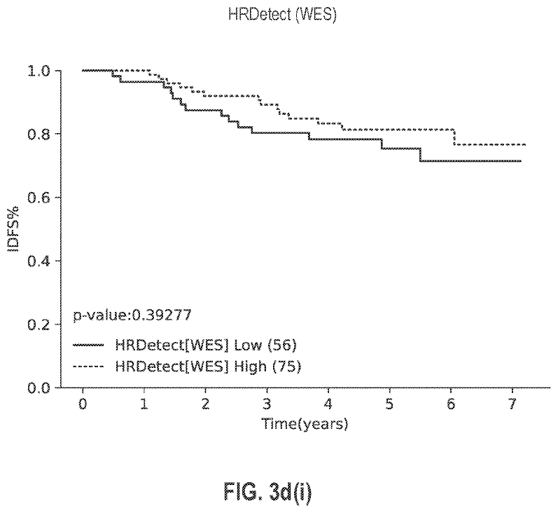

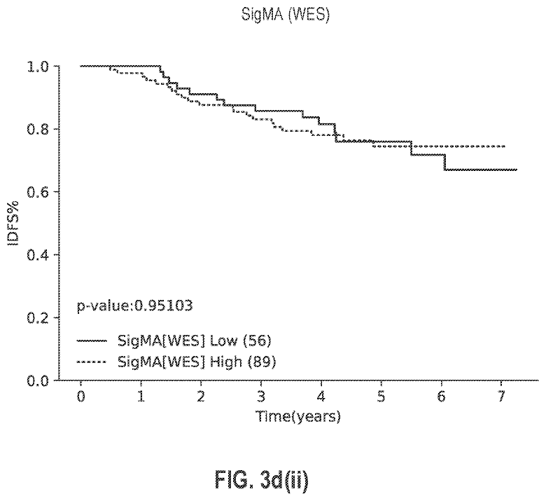

To assess the predictive capability of the WGS HRProfiler model on an independent WGS breast dataset, the HRD status was determined using the WGS HRProfiler model for 237 triple negative breast cancers (TNBCs) with known HRD and HRP annotations as well as known response to prior platinum treatmenty²³. Then, the performance of HRProfiler was compared to the performances of HRDetect, CHORD, and SigMA. As in the prior WGS dataset, HRProfiler delivered comparable performance to the other tools at the WGS resolution (FIG. 3a). Similarly, from a clinical endpoint perspective, all tools exhibited results showing comparable prognostic benefit based on disease-free survival (IDFS) for HRD classified patients with prior chemotherapy treatment (p-values<0.05; log-rank tests; FIG. 3b(i)-(iv)). To determine the predictive power and applicability of the WGS HRProfiler model on a lower genomic resolution, the genomic features of 237 triple negative breast cancers were down-sampled to exome-resolution first and the previously pre-trained WGS model of HRDetect, WES models applied for HRProfiler, and SigMA. CHORD was not used on this data as the tool only supports whole-genome sequenced samples¹⁷. HRProfiler was able to better separate HRD and HRP samples from the down-sampled dataset (FIG. 3c). Importantly, HRProfiler was the only tool that was able to achieve significant stratification based on IDFS across HRD and HRP samples (p-value: 0.009; log-rank test; FIG. 3d(i)-(iii)).

### Example 6

### Training and Validating HRProfiler to Predict HRD Status from Ovarian Samples

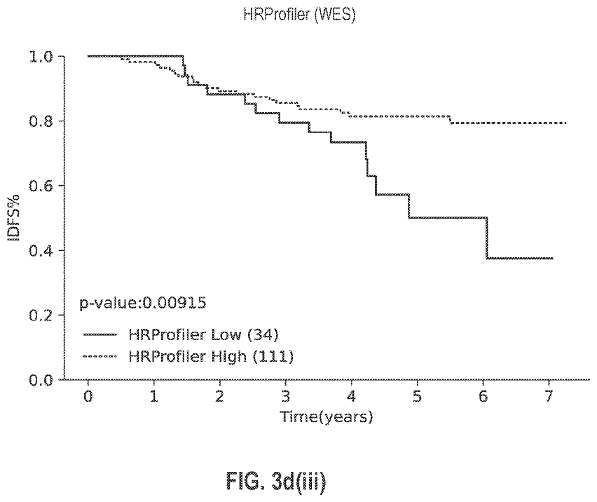

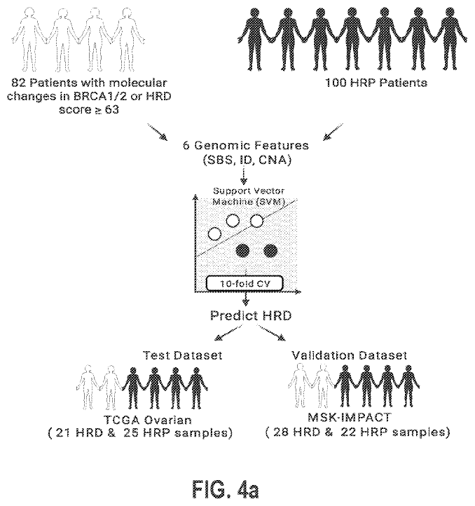

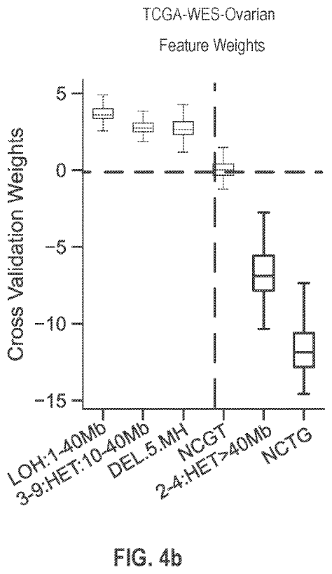

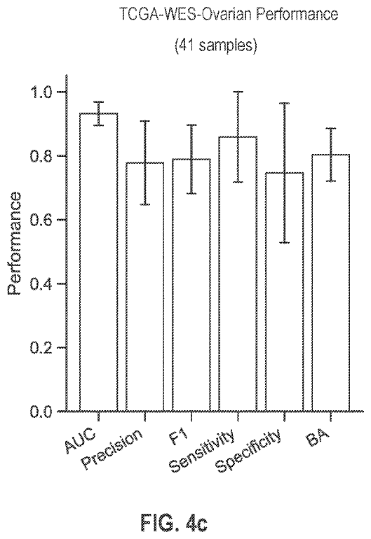

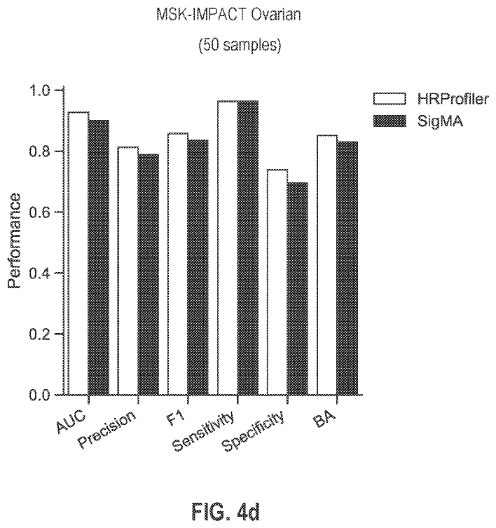

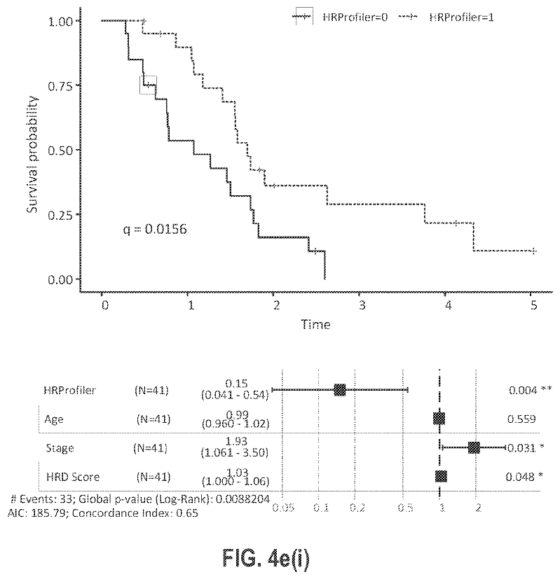

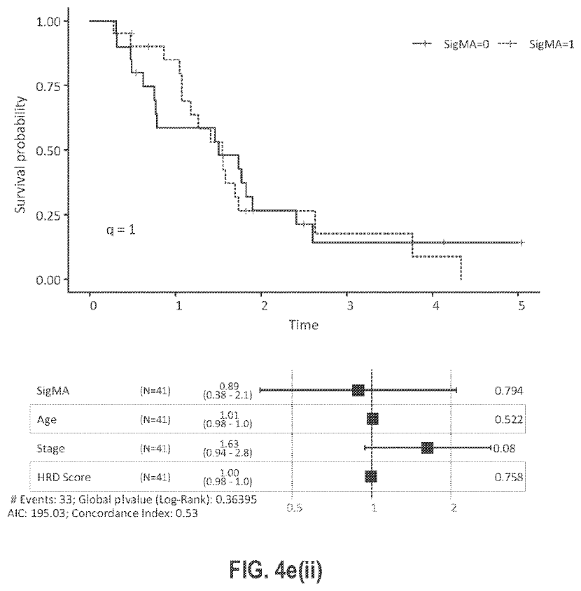

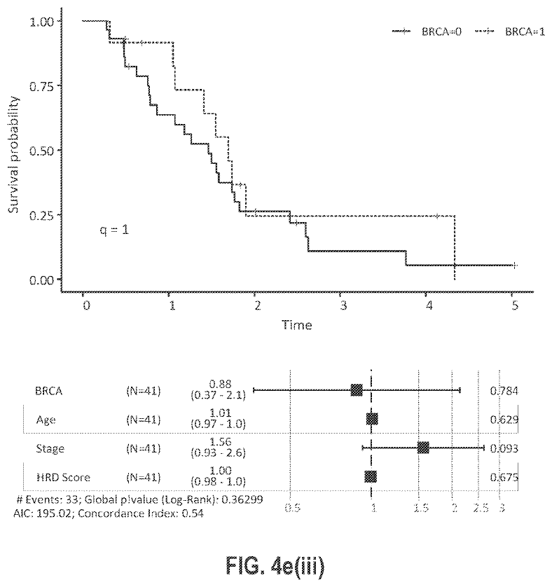

To determine if the defined genomic features can be generalized to other HRD-associated cancers, a tissue-specific model for ovarian cancer was trained using 182 TCGA ovarian exome patients (TCGA-WES-Ovarian) that comprised of 82 HRD and 100 HRP patients (FIG. 4a). For training purposes, patients were classified as HRD based on genomic alterations in BRCA1 and BRCA2 or an HRD score of at least 63. Ten-fold cross validation were conducted to determine the feature weights for the trained model (FIG. 4b). Features with positive weights (LOH: 1-40 Mb, DEL.5.MH, 3-9: HET: 10-40 Mb, and N[C>G]T) were enriched in HRD samples, whereas, features with negative weights (N[C>T]G and 2-4:Het:>40 Mb) were enriched in HRP samples. The model's performance was tested by generating 100 training and test datasets by random sampling based on 80/20 split between the training and the testing dataset. HRProfiler had an average AUC of 0.93 and an F1-score of 0.78 across 100 test datasets (FIG. 4c). To validate ovarian-specific HRProfiler model performance on an independent, external dataset, the model was applied to predict HRD status for 50 MSK-IMPACT ovarian samples with known HRD annotations and its performance was comparable to SigMA (FIG. 4d). To assess if HRProfiler can serve as a prognostic biomarker, it was determined that if there is a statistically significant difference in survival between HRD and HRP patients in the held-out test dataset. Progression Free Interval (PFI) analysis revealed better survival for HRD patients stratified based on HRProfiler (q-value=0.0156; Cox proportional hazards ratio) but not based on SigMA (q-value=1; Cox proportional hazards ratio) and BRCA1/2 mutation status (q-value=1; Cox proportional hazards ratio) in held out TCGA ovarian patients pre-treated with platinum therapy after correcting for age, clinical stage and HRD score (FIG. 4e(i)-(iii)).

### Example 7

### Online Methods: Data Sets

In the present disclosure, published datasets were used for feature engineering, model development, and validation at both whole-genome and whole-exome sequencing resolutions. For the analysis at the whole-genome resolution, CaVEman mutation calls and ASCAT allele-specific copy number calls were used for 371 samples from the 560 Breast Dataset²¹ [ftp://ftp.sanger.ac.uk/pub/cancer/Nik-ZainalEtAI-560BreastGenomes/]. Additional WGS datasets used in this study included the 237 Triple Negative Breast (TNBC) samples part of the SCAN-B trial²³. CaVEman mutation calls and ASCAT copy number calls for the 237 TNBC samples were downloaded from: https://data.mendeley.com/datasets/2mn4ctdpxp/. For the PCAWG dataset, consensus mutation and copy number calls were downloaded from the ICGC data portal: https://dcc.icgc.org/releases/PCAWG.

For the analysis at whole-exome resolution, TCGA dataset were utilized. The catalogues of somatic mutations were downloaded from GDC, and allele-specific exome copy number calls were derived in house. MSK-IMPACT exome 109 breast and 50 ovarian samples were downloaded from dbGaP and processed in house using the EVC pipeline.

### Example 8

### HRD Definition

Given the lack of clinical response to PARP inhibitors or platinum therapies available for majority of the data, a pseudo-ground truth for HRD was derived, which is based on the presence of germline or somatic alterations in BRCA1, and BRCA2, or an HRD score of at least 42 for breast and 63 for ovarian patients.

### Example 9

### Feature Engineering for Predicting HRD

To identify significantly enriched features in HRD and HRP samples, the average mutational profiles were generated based on proportions across the 96 mutation, 83 indel, and 48 copy number contexts. To determine significant channels at every resolution, a Fisher's exact test was conducted to determine if there is any significant difference in the average proportion of a given channel across HRD and HRP samples. Significant channels were identified at all the contexts if their logz fold-change (FC) is greater than 0.75 for WGS samples and 0.25 for WES samples, and their −log₁₀(p-adjusted value) is greater than 3. Similar workflow was adopted for both whole-genome and whole exome samples and only channels significantly enriched across both were considered for the feature engineering process. At the single base resolution, A[C>T]G, C[C>T]G, G[C>T]G, T[C>T]G channels are consistently enriched across HRP samples in both whole-genome and exome datasets and have an overlapping/similar mutational context, therefore, these 4 channels were combined into a single feature termed N[C>T]G, where N represents any of the 4 nucleotide bases(A/C/T/G). Similarly, A[C>G]T, C[C>G]T, G[C>G]T are all significant channels enriched in HRD samples and were combined into a single feature N[C>G]T, where N represents all possible nucleotide bases. At the indel resolution, 5:Del:M:1, 5:Del:M:2, 5:Del:M:3, 5:Del:M:4, 5:Del:M:5 are significant channels that all represent varying lengths of microhomology sequences at relatively large deletion sites where the length of the deletion is at least 5 base pairs. These indel channels were combined into a single feature: DEL.5.MH, where DEL.5 presents deletions of length at least 5 bp and MH represent microhomology sequences. At the copy number resolution, multiple significant channels for Loss of Heterozygosity (LOH) were identified that represented LOH segments of sizes between 1 to 40 Mb. These were combined into a single feature LOH.1.40 Mb. Similar approach was applied to aggregate significant copy number channels for diploid/genome-doubled copy number segments into a single feature 2-4:HET:>40 Mb that accounts for segments that have a total copy number state between 2-4 and their size is at least 40 Mb. Lastly, significant copy number channels for amplification events were combined into a single feature: 3-9: HET: 10-40 Mb, where 3-9 represents the segments with a total copy number state of at least 3 and segment sizes between 10 to 40 Mb.

### Example 10

### Model Development and Performance at WGS

To train a model for predicting HRD at WGS resolution, samples from the 560 Breast dataset were used. Only 371/560 samples that were labelled as evaluated in the HRDetect publication were considered. The six features derived from the feature engineering step were extracted from the 371 samples and were normalized using min max normalization. The initial training was based on 311 breast samples that comprised of 121 HRD and 190 HRP samples. Next, 10-fold cross validations were conducted to tune for hyper-parameters and obtain feature weights from the model. The model's performance was tested on the entire 371 breast dataset and an HRD probability threshold of 0.3 was used to classify a sample as HRD. The final HRD model was trained on all 371 breast samples using a linear kernel support vector machine (SVM) with L1 regularization and tuned hyperparameters. To validate the model on an external dataset, we predicted HRD probabilities for the 237 Triple Negative Breast (TNBC) samples and evaluated its performance against the ground truth based on molecular changes in the HR pathway or an HRD score of at least 42. The performance of the model was assessed using conventional machine learning metrics such as AUC, Sensitivity, Specificity, Precision, Balanced Accuracy (BA), and F1. To compare the performance of HRProfiler with other tools, HRD probabilities were determined for the 237 TNBC samples using the default settings for HRDetect, CHORD and SigMA.

### Example 11

### Model Development and Performance at WES

To train a model for predicting HRD at WES resolution, samples from the TCGA breast dataset were used. Only 736 samples that had HRD annotations were used for both training and testing. The six features derived from the feature engineering step were extracted as proportions, except for DEL.5.MH, which was extracted as absolute counts. Next, all features were scaled individually by min max normalization. The initial training was based on 671 breast samples that comprised of 157 HRD and 514 HRP samples. Next, 10-fold cross validations were conducted to tune for hyper-parameters and obtain feature weights from the model. The model's performance was tested on the 65 breast samples that were sequenced at both whole-genome and exome resolution. Samples with an HRD probability at least 0.1 were considered as HRD. To validate the model on an external dataset, HRD probabilities were predicted for 109 MSK-IMPACT breast exome samples and evaluated the model's performance against the ground truth based on molecular changes in the HR pathway or an HRD score of at least 42. The performance of the model was assessed using conventional machine learning metrics such as AUC, Sensitivity, Specificity, Precision, Balanced Accuracy (BA), and F1. To compare the performance of HRProfiler with other tools, HRD probabilities were determined for the same samples using the default settings for SigMA. The WES model was also applied to the down-sampled 237 TNBC samples and its performance was compared with that of other tools, including HRDetect and SigMA using the default WGS and WES pre-trained models respectively. The exome features for the 237 TNBC samples were derived by down-sampling the available SNP6 ASCAT copy number calls to segments that spanned the exonic regions. The mutation and indel calls were down sampled to exome resolution using SigProfilerMatrixGenerator.

### Example 12

### Survival Analysis and Statistical Analysis

The survival analysis was conducted using the Kaplan Meier (KM) and Cox Proportional-Hazards Model (COXPH) functions from the survminer and survival packages in R. Interval Disease Free Survival (IDFS) was used to evaluate the prognostic benefit in patients treated with chemotherapy from the 237 TNBC dataset. Progression Free Interval (PFI) endpoint was used to evaluate the survival trends for TCGA ovarian cancer patients treated with platinum therapy.

All statistical analysis were conducted in python using the scikit-learn package in python. All p-values were corrected for multiple hypothesis testing using Benjamini-Hochberg where needed.

In summary, the present technology provides a machine learning approach termed HRProfiler that uses a minimum set of six genomic features to predict homologous recombination deficiency across both whole-genome and whole-exome sequencing data. HRProfiler has similar performance to current tools when applied to whole-genome and outperforms all existing approaches when applied to whole-exome sequencing. HRProfiler incorporates features enriched in both HRD and HRP samples, which are not considered in current methods as they generally focus on mutation types enriched exclusively in HRD samples¹⁷⁻¹⁹. HRProfiler circumvents the need for structural variations and mutational signature extraction, which could be unreliable when using sparse datasets derived from whole-exome and targeted-panel sequencing²⁷. The use of a single mutational signatures, such as SBS326, is not reliable for accurate HRD prediction. SBS3 is a flat mutational signature with a high probability of misassigned mutations in a cancer genome enriched for other correlated flat mutational signatures such as SBS5 and SBS40. The use of N[C>T]G and N[C>G]T as HRP-specific features serves as a reliable alternative to SBS3 and overcomes the problems associated with the use of flat mutational signatures as a biomarker at the exome resolution.

The application of HRProfiler across both breast and ovarian cancers outlines the generalizability of features across different cancer types. Overall, the machine learning approach disclosed herein bridges the gap in using the molecular phenotypic footprint of failed DNA repair processes as clinical biomarkers for the reliable stratification of patients sensitive to PARP inhibitors and/or platinum therapies.

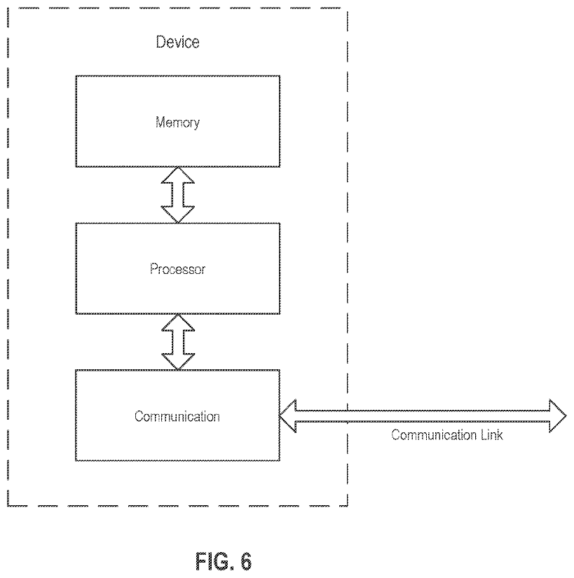

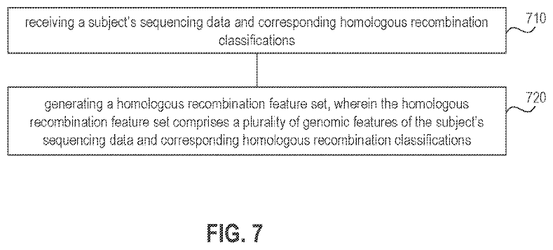

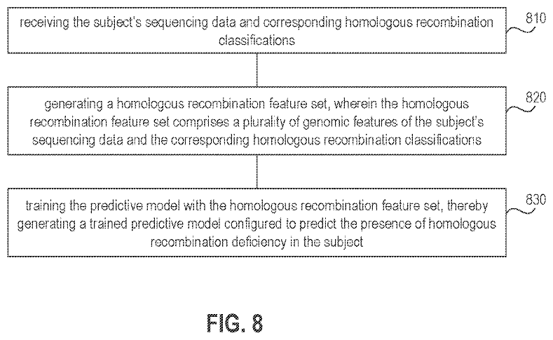

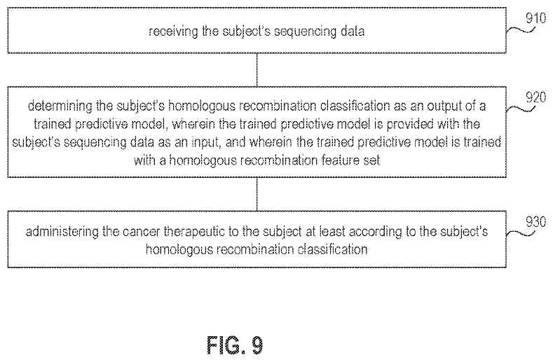

It is understood that the various disclosed embodiments may be implemented individually, or collectively, using devices comprised of various components, electronics hardware and/or software modules and components. These devices, for example, may comprise a processor, a memory unit, an interface that are communicatively connected to each other, and may range from desktop and/or laptop computers, to mobile devices and the like. The processor and/or controller can perform various disclosed operations based on execution of program code that is stored on a storage medium. The processor and/or controller can, for example, be in communication with at least one memory and with at least one communication unit that enables the exchange of data and information, directly or indirectly, through the communication link with other entities, devices and networks. The communication unit may provide wired and/or wireless communication capabilities in accordance with one or more communication protocols, and therefore it may comprise the proper transmitter/receiver antennas, circuitry and ports, as well as the encoding/decoding capabilities that may be necessary for proper transmission and/or reception of data and other information. FIG. 6 illustrates one example of such a device that includes at least one processor and/or controller, at least one memory unit that is in communication with the processor, and at least one communication unit that enables the exchange of data and information, directly or indirectly, through the communication link with other entities, devices, databases and networks.

Various information and data processing operations described herein may be implemented in one embodiment by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Therefore, the computer-readable media that is described in the present application comprises non-transitory storage media. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known components and functions have not been shown or described in detail to avoid unnecessarily obscuring the description of the embodiments of the technology. Where the context permits, singular or plural terms may also include the plural or singular term, respectively. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

### REFERENCES

1 Ceccaldi, R., Rondinelli, B. & D'Andrea, A. D. in Trends in Cell Biology Vol. 26 52-64 (Elsevier Ltd, 2016).
2 Konstantinopoulos, P. A., Ceccaldi, R., Shapiro, G. I. & D'Andrea, A. D. Homologous Recombination Deficiency: Exploiting the Fundamental Vulnerability of Ovarian Cancer. Cancer Discov 5, 1137-1154 (2015). https://doi.org:10.1158/2159-8290.CD-15-0714
3 Kasi, A., Al-Jumayli, M., Park, R., Baranda, J. & Sun, W. in Journal of Pancreatic Cancer Vol. 6 107-115 (2020).
4 Abida, W. et al. Rucaparib in Men With Metastatic Castration-Resistant Prostate Cancer Harboring a BRCA1 or BRCA2 Gene Alteration. J Clin Onco/38, 3763-3772 (2020). https://doi. org:10.1200/JCO.20.01035
5 de Bono, J. et al. Olaparib for Metastatic Castration-Resistant Prostate Cancer. N Engl J Med 382, 2091-2102 (2020). https://doi.org:10.1056/NEJMoa1911440
6 Moore, K. et al. Maintenance Olaparib in Patients with Newly Diagnosed Advanced Ovarian Cancer. N Engl J Med 379, 2495-2505 (2018). https://doi.org:10.1056/NEJMoa1810858
7 Tutt, A. et al. in Nature Medicine Vol. 24 628-637 (Springer US, 2018).
8 Curtin, N. J. & Szabo, C. in Nature Reviews Drug Discovery Vol. 19 711-736 (Springer US, 2020).
9 Wang, D. & Lippard, S. J. Cellular processing of platinum anticancer drugs. Nat Rev Drug Discov 4, 307-320 (2005). https://doi.org:10.1038/nrd1691
10 Abkevich, V. et al. in British Journal of Cancer Vol. 107 1776-1782 (2012).
11 Melinda, L. T. et al. in Clinical Cancer Research Vol. 22 3764-3773 (2016).
12 Birkbak, N. J. et al. in Cancer Discovery Vol. 2 366-375 (2012).
13 Miller, R. E. et al. ESMO recommendations on predictive biomarker testing for homologous recombination deficiency and PARP inhibitor benefit in ovarian cancer. Ann Onco/31, 1606-1622 (2020). https://doi.org:10.1016/j. annonc.2020.08.2102
14 Popova, T. et al. in Cancer Research Vol. 72 5454-5462 (2012).
15 How, J. A. et al. in Cancers Vol. 13 1-18 (2021).
16 Takaya, H., Nakai, H., Takamatsu, S., Mandai, M. & Matsumura, N. in Scientific Reports Vol. 10 1-8 (2020).
17 Davies, H. et al. in Nature Medicine Vol. 23 517-525 (Nature Publishing Group, 2017).
18 Nguyen, L., W. M. Martens, J., Van Hoeck, A. & Cuppen, E. in Nature Communications Vol. 11 1-12 (2020).
19 Gulhan, D. C., Lee, J. J. K., Melloni, G. E. M., Cortes-Ciriano, I. & Park, P. J. Detecting the mutational signature of homologous recombination deficiency in clinical samples. Nature Genetics 51, 912-919 (2019). https://doi.org:10.1038/s41588-019-0390-2
20 Alexandrov, L. B. et al. Signatures of mutational processes in human cancer. Nature 500, 415-421 (2013). https://doi.org:10.1038/nature12477
21 Nik-Zainal, S. et al. in Nature Vol. 534 47-54 (Nature Publishing Group, 2016).
22 Alexandrov, L. B. et al. The repertoire of mutational signatures in human cancer. Nature 578, 94-101 (2020). https://doi.org:10.1038/s41586-020-1943-3
23 Staaf, J. et al. in Nature Medicine Vol. 25 (Springer US, 2019).
24 Zehir, A. et al. in Nature Medicine Vol. 23 703-713 (2017).
25 Van Allen, E. M. et al. in Nature Medicine Vol. 20 682-688 (Nature Publishing Group, 2014).
26 Alexandrov, L. B. et al. in Nature Vol. 500 415-421 (2013).
27 Alexandrov, L. B. et al. in Nature Vol. 578 94-101 (2020).
28 Steele, C. D. et al. in Nature Vol. 606 984-991 (Springer US, 2022).
29 Steele, C. D. et al. Signatures of copy number alterations in human cancer. Nature 606, 984-991 (2022). https://doi.org: 10.1038/s41586-022-04738-6
30 Gao, G. F. et al. Before and After: Comparison of Legacy and Harmonized TCGA Genomic Data Commons' Data. Cell Syst 9, 24-34 e10 (2019). https://doi.org:10.1016/j.cels.2019.06.006
31 Pettitt, S. J. et al. Clinical brca 1/2 reversion analysis identifies hotspot mutations and predicted neoantigens associated with therapy resistance. Cancer Discovery 10, 1475-1488 (2020). https://doi.org:10.1158/2159-8290.CD-19-1485
32 Marquard, A. M. et al. Pan-cancer analysis of genomic scar signatures associated with homologous recombination deficiency suggests novel indications for existing cancer drugs. Biomarker Research 3, 1-10 (2015). https://doi.org: 10.1186/s40364-015-0033-4

1. A method of generating a homologous recombination feature set, the method comprising:
(a) receiving a subject's sequencing data and corresponding homologous recombination classifications; and
(b) generating a homologous recombination feature set, wherein the homologous recombination feature set comprises a plurality of genomic features of the subject's sequencing data and corresponding homologous recombination classifications.

2. The method of claim 1, wherein the sequencing data comprises whole-genome sequencing data, whole-exome sequencing data, a fraction thereof, or any combination thereof.

3. The method of claim 1 or 2, wherein the homologous recombination feature set comprises: a total number and a proportion of deletions at microhomologies features of the sequencing data, a total number and a proportion of genomic segments with loss of heterozygosity features of the sequencing data, a total number and a proportion of heterozygous genomic segments features of the sequencing data, a total number and a proportion of C:G>T:A single base substitutions at a 5′-NpCpG-3′ contexts features of the sequencing data, a total number and a proportion of C:G>G:C single base substitutions at a 5′-NpCpT-3′ contexts features of the sequencing data, or any combination thereof.

4. The method of claim 3, wherein the total number and the proportions of genomic segments with loss of heterozygosity comprise a size from about 1 to about 40 megabases with at least 1 copy of the genomic segments with loss of heterozygosity.

5. The method of claim 3, wherein the total number and the proportions of heterozygous genomic segments comprise a size from about 3 to about 40 megabases with 3 to 9 copies of each heterozygous genomic segment of the heterozygous genomic segments.

6. The method of claim 3, wherein the total number and the proportions of heterozygous genomic segments comprise a size of at least 40 megabases with 2 to 4 copies of each heterozygous genomic segment of the heterozygous genomic segments.

7. The method of claim 3, wherein the total number and the proportions of deletions at microhomologies comprise a size of at least 5 base-pairs.

8. The method of any one of claims 1-7, wherein the homologous recombination classification comprises: homologous recombination deficiency positive or homologous recombination deficiency negative.

9. The method of any one of claims 1-8, wherein the subject's sequencing data comprises retrospective clinical trial sequencing data of a patient that participated in a clinical trial, wherein the patient is the same as or different from the subject.

10. A method of training a predictive model configured to predict a presence of homologous recombination deficiency in a subject, the method comprising:
(a) receiving the subject's sequencing data and corresponding homologous recombination classifications;
(b) generating a homologous recombination feature set, wherein the homologous recombination feature set comprises a plurality of genomic features of the subject's sequencing data and the corresponding homologous recombination classifications; and
(c) training the predictive model with the homologous recombination feature set, thereby generating a trained predictive model configured to predict the presence of homologous recombination deficiency in the subject.

11. The method of claim 10, wherein the training comprises linear kernel support vector machine (SVM) with L1 regularization.

12. The method of claim 10 or 11, wherein the predictive model comprises a random forest predictive model, a naïve Bayes classifier predictive model, a support vector machine predictive model, a logistic regression predictive model, or any combination thereof.

13. The method of any one of claims 10-12, wherein the predictive model is configured to predict the presence of homologous recombination deficiency in the subject with an accuracy of at least 60%, at least 65%, at least 70%, at least 75%, at least 80%, at least 85%, at least 90%, at least 95%, or at least 99%.

14. The method of any one of claims 10-13, wherein the predictive model is configured to predict the presence of homologous recombination deficiency in the subject with a precision of at least 60%, at least 65%, at least 70%, at least 75%, at least 80%, at least 85%, at least 90%, at least 95%, or at least 99%.

15. The method of any one of claims 10-14, wherein the predictive model is configured to predict the presence of homologous recombination deficiency in the subject with a F1 of at least 60%, at least 65%, at least 70%, at least 75%, at least 80%, at least 85%, at least 90%, at least 95%, or at least 99%.

16. The method of any one of claims 10-15, wherein the predictive model is configured to predict the presence of homologous recombination deficiency in the patient with a sensitivity of at least 60%, at least 65%, at least 70%, at least 75%, at least 80%, at least 85%, at least 90%, at least 95%, or at least 99%.

17. The method of any one of claims 10-16, wherein the predictive model is configured to predict the presence of homologous recombination deficiency in the patient with a specificity of at least 60%, at least 65%, at least 70%, at least 75%, at least 80%, at least 85%, at least 90%, at least 95%, or at least 99%.

18. The method of any one of claims 10-17, wherein the predictive model is configured to predict the presence of homologous recombination deficiency in the patient with a balanced accuracy of at least 60%, at least 65%, at least 70%, at least 75%, at least 80%, at least 85%, at least 90%, at least 95%, or at least 99%.

19. The method of any one of claims 10-18, wherein the sequencing data comprises whole-genome sequencing data, whole-exome sequencing data, a fraction thereof, or any combination thereof.

20. The method of any one of claims 10-19, wherein the homologous recombination feature set comprises: a total number and proportions of deletions at microhomologies features of the sequencing data, a total number and proportions of genomic segments with loss of heterozygosity features of the sequencing data, a total number and proportions of heterozygous genomic segments features of the sequencing data, a total number and proportions of C:G>T:A single base substitutions at a 5′-NpCpG-3′ contexts features of the sequencing data, a total number and a proportion of C:G>G:C single base substitutions at a 5′-NpCpT-3′ contexts features of the sequencing data, or any combination thereof.

21. £ The method of claim 20, wherein the total number and the proportions of genomic segments with loss of heterozygosity comprise a size from about 1 to about 40 megabases with at least 1 copy of the genomic segments with loss of heterozygosity.

22. The method of claim 20, wherein the total number and the proportions of heterozygous genomic segments comprise a size from about 3 to about 40 megabases with 3 to 9 copies of each heterozygous genomic segment of the heterozygous genomic segments.

23. The method of claim 20, wherein the total number and the proportions of heterozygous genomic segments comprise a size of at least 40 megabases with 2 to 4 copies of each heterozygous genomic segment of the heterozygous genomic segments.

24. The method of claim 20, wherein the total number and the proportions of deletions at microhomologies comprise a size of at least 5 base-pairs.

25. The method of any one of claims 10-24, wherein the homologous recombination classification comprises: homologous recombination deficiency positive or homologous recombination deficiency negative.

26. The method of any one of claims 10-25, wherein the subject's sequencing data comprises retrospective clinical trial sequencing data of a patient that participated in a clinical trial, wherein the patient is the same as or different from the subject.

27. A method of administering a cancer therapeutic to a subject, the method comprising:
(a) receiving the subject's sequencing data;
(b) determining the subject's homologous recombination classification as an output of a trained predictive model, wherein the trained predictive model is provided with the subject's sequencing data as an input, and wherein the trained predictive model is trained with a homologous recombination feature set; and
(c) administering the cancer therapeutic to the subject at least according to the subject's homologous recombination classification.

28. The method of claim 27, wherein the cancer therapeutic comprises at least one selected from the group consisting of platinum therapies and poly (ADP-ribose) polymerase (PARP) inhibitors.

29. The method of claim 28, wherein the PARP inhibitors comprise Talazoparib, Olaparib, Niraparib, Rucaparib, Veliparib, or any combination thereof.

30. The method of claim 28, wherein the platinum therapies comprise Cisplatin, Oxaliplatin, Carboplatin, or any combination thereof.

31. The method of any one of claims 27-30, wherein the cancer therapeutic causes inter-strand breaks of genomic molecules of the subject's cells, leading to p53-initiated apoptosis.

32. The method of any one of claims 27-31, wherein the subject is suspected of having cancer.

33. The method of claim 32, wherein the cancer is at least one selected from the group consisting of breast cancer, ovarian cancer, prostate cancer, pancreatic cancer, and sarcoma.

34. The method of any one of claims 27-33, wherein the trained predictive model comprises a predictive model trained by a linear kernel support vector machine (SVM) with L1 regularization.

35. The method of any one of claims 27-34, wherein the trained predictive model is configured to determine the subject's homologous recombination classification with an accuracy of at least 60%, at least 65%, at least 70%, at least 75%, at least 80%, at least 85%, at least 90%, at least 95%, or at least 99%.

36. The method of any one of claims 27-35, wherein the trained predictive model is configured to determine the subject's homologous recombination classification with a precision of at least 60%, at least 65%, at least 70%, at least 75%, at least 80%, at least 85%, at least 90%, at least 95%, or at least 99%.

37. The method of any one of claims 27-36, wherein the trained predictive model is configured to determine the subject's homologous recombination classification with a F1 of at least 60%, at least 65%, at least 70%, at least 75%, at least 80%, at least 85%, at least 90%, at least 95%, or at least 99%.

38. The method of any one of claims 27-37, wherein the trained predictive model is configured to determine the subject's homologous recombination classification with a sensitivity of at least 60%, at least 65%, at least 70%, at least 75%, at least 80%, at least 85%, at least 90%, at least 95%, or at least 99%.

39. The method of any one of claims 27-38, wherein the trained predictive model is configured to determine the subject's homologous recombination classification with a specificity of at least 60%, at least 65%, at least 70%, at least 75%, at least 80%, at least 85%, at least 90%, at least 95%, or at least 99%.

40. The method of any one of claims 27-39, wherein the trained predictive model is configured to determine the subject's homologous recombination classification with a balanced accuracy of at least 60%, at least 65%, at least 70%, at least 75%, at least 80%, at least 85%, at least 90%, at least 95%, or at least 99%.

41. The method of any one of claims 27-40, wherein the sequencing data comprises whole-genome sequencing data, whole-exome sequencing data, any fraction thereof, or any combination thereof.

42. The method of any one of claims 27-41, wherein the homologous recombination feature set comprises genomic features.

43. The method of claim 42, wherein the genomic features comprise: a total number and proportions of deletions at microhomologies features of the sequencing data, a total number and proportions of genomic segments with loss of heterozygosity features of the sequencing data, a total number and proportions of heterozygous genomic segments features of the sequencing data, a total number and proportions of C:G>T:A single base substitutions at a 5′-NpCpG-3′ contexts features of the sequencing data, a total number and a proportion of C:G>G:C single base substitutions at a 5′-NpCpT-3′ contexts features of the sequencing data, or any combination thereof.

44. The method of claim 43, wherein the total number and the proportion of genomic segments with loss of heterozygosity comprise a size from about 1 to about 40 megabases with at least 1 copy of the genomic segments with loss of heterozygosity.

45. The method of claim 43, wherein the total number and the proportion of heterozygous genomic segments comprise a size from about 3 to about 40 megabases with 3 to 9 copies of each heterozygous genomic segment of the heterozygous genomic segments.

46. The method of claim 43, wherein the total number and the proportion of heterozygous genomic segments comprise a size of at least 40 megabases with 2 to 4 copies of each heterozygous genomic segment of the heterozygous genomic segments.

47. The method of claim 43, wherein the total number and the proportion of deletions at microhomologies comprise a size of at least 5 base-pairs.

48. The method of any one of claims 27-47, wherein the homologous recombination classification comprises: homologous recombination deficiency positive or homologous recombination deficiency negative.

49. The method of any one of claims 27-48, wherein the subject's sequencing data comprises retrospective clinical trial sequencing data of a patient that participated in a clinical trial, wherein the patient is the same as or different from the subject.

50. A computer system configured to output a homologous recombination classification of a subject, the computer system comprises:
(a) one or more processors;
(b) non-transient computer readable storage medium including software, wherein the software comprises executable instructions that, as a result of execution, cause the one or more processors of the computer system to:
(i) receive the subject's sequencing data; and
(ii) output the subject's homologous recombination classification as an output of a trained predictive model when the trained predictive model is provided with the subject's sequencing data as an input, wherein the trained predictive model is trained with a homologous recombination feature set.

51. The computer system of claim 50, wherein the software comprises determining a cancer therapeutic at least according to the subject's homologous recombination classification.

52. The computer system of claim 51, wherein the cancer therapeutic comprises at least one selected from the group consisting of platinum therapies and poly (ADP-ribose) polymerase (PARP) inhibitors.

53. The computer system of claim 52, wherein the PARP inhibitors comprise Talazoparib, Olaparib, Niraparib, Rucaparib, Veliparib, or any combination thereof.

54. The computer system of claim 52, wherein the platinum therapies comprise Cisplatin, Oxaliplatin, Carboplatin, or any combination thereof.

55. The computer system of any one of claims 51-54, wherein the cancer therapeutic causes inter-strand breaks of genomic molecules of the subject's cells, leading to p53-initiated apoptosis.

56. The computer system of any one of claims 50-55, wherein the subject is suspected of having cancer.

57. The computer system of claim 56, wherein the cancer is at least one selected from the group consisting of breast cancer, ovarian cancer, prostate cancer, pancreatic cancer, and sarcoma.

58. The computer system of any one of claims 50-57, wherein the trained predictive model comprises a predictive model trained by a linear kernel support vector machine (SVM) with L1 regularization.

59. The computer system of any one of claims 50-58, wherein the sequencing data comprises whole-genome sequencing data, whole-exome sequencing data, a fraction thereof, or any combination thereof.

60. The computer system of any one of claims 50-59, wherein the homologous recombination feature set comprises genomic features.

61. The computer system of claim 60, wherein the genomic features comprise: a total number and a proportion of deletions at microhomologies features of the sequencing data, a total number and a proportion of genomic segments with loss of heterozygosity features of the sequencing data, a total number and a proportion of heterozygous genomic segments features of the sequencing data, a total number and a proportion of C:G>T:A single base substitutions at a 5′-NpCpG-3′ contexts features of the sequencing data, a total number and a proportion of C:G>G:C single base substitutions at a 5′-NpCpT-3′ contexts features of the sequencing data, or any combination thereof.

62. The computer system of claim 61, wherein the total number and the proportions of genomic segments with loss of heterozygosity comprise a size from about 1 to about 40 megabases with at least 1 copy of the genomic segments with loss of heterozygosity.

63. The computer system of claim 61, wherein the total number and the proportions of heterozygous genomic segments comprise a size from about 3 to about 40 megabases with 3 to 9 copies of each heterozygous genomic segment of the heterozygous genomic segments.

64. The computer system of claim 61, wherein the total number and the proportions of heterozygous genomic segments comprise a size of at least 40 megabases with 2 to 4 copies of each heterozygous genomic segment of the heterozygous genomic segments.

65. The computer system of claim 61, wherein the total number and the proportions of deletions at microhomologies comprise a size of at least 5 base-pairs.

66. The computer system of any one of claims 50-65, wherein the homologous recombination classification comprises: homologous recombination deficiency positive or homologous recombination deficiency negative.

67. The computer system of any one of claims 50-66, wherein the trained predictive model is configured to output the subject's homologous recombination classification with an accuracy of at least 60%, at least 65%, at least 70%, at least 75%, at least 80%, at least 85%, at least 90%, at least 95%, or at least 99%.

68. The computer system of any one of claims 50-67, wherein the trained predictive model is configured to output the subject's homologous recombination classification with a precision of at least 60%, at least 65%, at least 70%, at least 75%, at least 80%, at least 85%, at least 90%, at least 95%, or at least 99%.

69. The computer system of any one of claims 50-68, wherein the trained predictive model is configured to output the subject's homologous recombination classification with a F1 of at least 60%, at least 65%, at least 70%, at least 75%, at least 80%, at least 85%, at least 90%, at least 95%, or at least 99%.

70. The computer system of any one of claims 50-69, wherein the trained predictive model is configured to output the subject's homologous recombination classification with a sensitivity of at least 60%, at least 65%, at least 70%, at least 75%, at least 80%, at least 85%, at least 90%, at least 95%, or at least 99%.

71. The computer system of any one of claims 50-70, wherein the trained predictive model is configured to output the subject's homologous recombination classification with a specificity of at least 60%, at least 65%, at least 70%, at least 75%, at least 80%, at least 85%, at least 90%, at least 95%, or at least 99%.

72. £ The computer system of any one of claims 50-71, wherein the trained predictive model is configured to output the subject's homologous recombination classification with a balanced accuracy of at least 60%, at least 65%, at least 70%, at least 75%, at least 80%, at least 85%, at least 90%, at least 95%, or at least 99%.

73. The computer system of any one of claims 50-72, wherein the subject's sequencing data comprises retrospective clinical trial sequencing data of a patient that participated in a clinical trial, wherein the patient is the same as or different from the subject.
